(19) United States
(12) Patent Application Publication
(10) Pub. No.: US 2018/0355410 A1
(43) Pub. Date: Dec. 13, 2018

(54) DIAGNOSIS AND TREATMENT OF INFECTIOUS DISEASE

(71) Applicant: Cambridge Enterprise Limited, Cambridge (GB)

(72) Inventors: Helen Lee, Cambridge (GB); Michael Powell, Cambridge (GB)

(21) Appl. No.: 15/735,567

(22) PCT Filed: Jun. 17, 2016
(86) PCT No.: PCT/GB2016/051831

§ 371 Date: Dec. 11, 2017

(30) Foreign Application Priority Data
Jun. 19, 2015 (GB) 1510876.4
May 31, 2016 (GB) 1609529.1

(51) Int. Cl. C12Q 1/689; A61K 31/7052; A61K 31/545; A61K 31/496

(52) U.S. Cl. CPC C12Q 1/689; A61K 31/7052; C12Q 2600/106; A61K 31/496; A61K 31/545

(57) ABSTRACT

Methods are described for determining whether a subject suffering from, or suspected of suffering from, an infectious disease caused by a microbe is infected with a strain of the microbe that is susceptible to an antimicrobial agent, where there exist different strains of the microbe that are resistant to the antimicrobial agent. The methods comprise determining whether nucleic acid of the strain of the microbe infecting the subject comprises wild-type nucleotide sequence at a conserved nucleotide position at which mutation is associated with resistance to the antimicrobial agent in nucleic acid of the different resistant strains. The methods are particularly applicable for determining whether a subject suffering from, or suspected of suffering from, Gonorrhoea is infected with a strain of Neisseria gonorrhoeae that is susceptible to an antimicrobial agent. Kits for use in the methods are described, as well as methods for treatment of infectious disease. Methods for reducing the prevalence of resistance of microbes causing infectious disease to antimicrobial agents are also described.

This invention relates to methods for diagnosis and treatment of infectious disease, for example sexually transmitted disease, such as Gonorrhoea, and to kits for use in such methods. The invention also relates to methods for reducing the prevalence of resistance of microbes causing infectious disease to antimicrobial agents.

Neisseria gonorrhoeae is a gram-negative bacterium and the aetiological agent of Gonorrhoea, a sexually transmitted infection that is of significant public health concern. Infection with Gonorrhoea is on the increase. The World Health Organisation (WHO) estimates that there are 106 million new cases of Gonorrhoea among adults globally per annum, a 21% increase upon the rate of infection in 2005. Gonorrhoea infection is often asymptomatic in females (≥50% of cases). This is a significant issue, as undiagnosed infection can lead to endometritis and pelvic inflammatory disease, which can result in infertility or loss of life through ectopic pregnancy. Infection in males is more commonly symptomatic (in ≥90% of cases), with symptoms including epididymitis, penile discharge, swelling and pain. Extra-genital infection is common, particularly in men who have sex with men, however it can be found in heterosexuals as well, depending on sexual history. Extra-genital infections are frequently asymptomatic, but contribute significantly to the transmission of Gonorrhoea infection between sexual partners.

Diagnosis of infection with Gonorrhoea is critical to reduce complications and limit onward transmission. However, delays inherent in current clinical pathways, as a result of centralised Chlamydia/Gonorrhoea diagnosis, mean that significant numbers of symptomatic patients are treated empirically according to their sexual history and symptoms in the absence of a positive diagnosis. Given that Gonorrhoea infection shares symptoms with a number of other sexually transmitted infections, overtreatment is a significant issue as symptomatic patients are treated with cocktails of several antibiotics, with no knowledge of the aetiology of infection. Such injudicious antibiotic use has been a significant contributing factor to the development of antimicrobial resistance in Gonorrhoea, which has led to its evolution to superbug status.

Over time, a wide range of antibiotics has been used for the treatment of Gonorrhoea infection. However, N. gonorrhoeae has proven to be exceedingly adept at developing antimicrobial resistance mechanisms, even in the absence of antimicrobial selection pressure. Azithromycin, a chemical derivative of Erythromycin, was used for the treatment of Gonorrhoea since the early 1980s (Erythromycin was not sufficiently effective for treatment). However, the development of resistance to Azithromycin developed quickly following its implementation and it is no longer used in antibiotic mono-therapy. Ciprofloxacin was widely used to treat Gonorrhoea infection from the mid-1980s. Initially, low doses were used but reduced susceptibility was observed by 1990. The minimum recommended dose was increased. However, resistance developed and spread quickly to the point where Ciprofloxacin had been abandoned as a treatment option by the mid-2000s. Two extended spectrum Cephalosporins (ESCs) have been widely used for treatment of Gonorrhoea infection: Cefixime and Ceftriaxone. Cefixime was the only oral ESC that was recommended by the World Health Organisation (WHO) for first-line therapy as it met the criteria for >95% cure rate. However, since reduced susceptibility to Cefixime was first observed in the late 2000s, recommended treatment was switched to Ceftriaxone and Azithromycin dual therapy.

The spectre of multi-drug resistant Gonorrhoea has been exacerbated by the liberal application of broad ranges of antibiotics to treat infection. Development of N. gonorrhoeae drug resistance has quickly followed the introduction of new antibiotics for treatment. A large proportion of Gonorrhoea types circulating worldwide are now only a few resistance markers away from developing into extensively drug resistant (XDR) strains (strains that are resistant to at least two commonly used antimicrobials). Indeed, two XDR strains have recently been identified in Japan and Europe; the H041 and F89 strains, respectively. Both strains are resistant to the extended spectrum Cephalosporin (ESC) Ceftriaxone, the last fully effective antimicrobial for Gonorrhoea treatment that can be used in mono-therapy.

In general, widespread use of particular antibiotics decreases their efficacy over time by promoting resistance through selective pressures. Conversely, decreasing the use of antibiotics could reduce prevalence of resistance to particular drugs over time. Thus, it is possible to slow the development of multi-drug resistant Gonorrhoea by limiting treatment to the narrowest range of antibiotics to which N. gonorrhoeae is susceptible. This is known as antimicrobial stewardship.

To facilitate reduction of antibiotic usage, it would be beneficial to be able to diagnose infection with Gonorrhoea rapidly, as this would reduce overtreatment as a result of syndromic management in symptomatic patients. For patients who are Gonorrhoea positive, it would be of significant benefit to know whether they are infected with a strain of N. gonorrhoeae that is susceptible or resistant to antibiotic treatment. This would guide prescription of the narrowest possible range of antibiotics to treat the infection effectively, thus extending the utility of the drugs that are currently available for the treatment of Gonorrhoea.

Nucleic acid amplification testing is now the ‘gold-standard’ for diagnosis of Gonorrhoea infection, so many clinical laboratories no longer receive samples that are suitable for determination of antibiotic susceptibility by Gonorrhoea culture techniques. Instead, surveillance of antimicrobial resistant strains is undertaken by random sampling of the population at ‘sentinel’ locations, where a full resistance profile is established by culture/agar dilution. This information is used to modify treatment guidelines, but may not be representative of the whole population. If molecular testing could be performed for each patient when they attend the clinic, effective treatments could be administered on a case-by-case basis, improving antimicrobial stewardship and treatment outcomes.

Currently, there is no reliable technology that allows for antibiotic susceptibility testing from non-culture specimens. Whilst a number of diagnostic assays for antimicrobial resistance determinants have been described in the literature (for penicillin, tetracycline, macrolides, fluoroquinolones and extended spectrum cephalosporins), their sensitivity/specificity is often suboptimal. There are also many different mutations that are responsible for antibiotic resistance, so it is not practicable to test for each different mutation to determine which resistant strain is present. There are currently no commercially available diagnostic platforms to establish the antibiotic resistance/susceptibility pattern of Gonorrhoea.

There is a need, therefore, for a test that can be used to determine rapidly whether a subject suffering from, or suspected of suffering from, Gonorrhoea is infected with a strain of N. gonorrhoeae that is susceptible or resistant to treatment with antibiotics.

Rather than carry out susceptibility testing by standard culture techniques, or carry out several different nucleic acid tests to determine the identity of the strain infecting the subject, the Applicant has appreciated that it is only necessary to determine whether the nucleic acid of the infecting strain comprises wild-type nucleotide sequence. In particular, it can simply be determined whether nucleic acid of the infecting strain comprises wild-type nucleotide sequence in a region of the nucleic acid with which mutation is known to be associated with resistance to the antimicrobial agent. If the subject is infected with a strain that comprises the wild-type nucleotide sequence, the subject can be administered with the antimicrobial agent as a monotherapy. If the subject is infected with a strain that does not comprise the wild-type sequence, the subject can be administered with a different antimicrobial agent, or with a combination of antimicrobial agents.

Use of such methods will limit the development and/or spread of antimicrobial resistance because the antimicrobial agent will be administered only to those subjects likely to be effectively treated by the antimicrobial agent, and not to those subjects infected with resistant strains.

The Applicant has recognised that, for each different antibiotic for which resistant strains of N. gonorrhoeae have developed, some positions in the nucleotide sequence are mutated in most, or almost all, strains that are resistant to that antibiotic. The Applicant has appreciated that it can readily be determined whether a particular strain is susceptible to an antibiotic by assessing whether one or more of these conserved nucleotide positions contain wild-type nucleotide sequence or not. This can be done by nucleic acid testing, without any need to perform Gonorrhoea culture techniques.

The Applicant has also recognised that such methods are applicable to other infectious diseases caused by microbes where there exist strains of the microbe that are susceptible to an antimicrobial agent, and different strains of the microbe that are resistant to the antimicrobial agent.

According to the invention, there is provided a method of determining whether a subject suffering from, or suspected of suffering from, an infectious disease caused by a microbe is infected with a strain of the microbe that is susceptible to an antimicrobial agent, wherein there exist one or more different strains of the microbe that are resistant to the antimicrobial agent, wherein the method comprises determining whether nucleic acid of the strain of the microbe infecting the subject comprises wild-type nucleotide sequence.

In particular, methods of the invention may comprise determining whether nucleic acid of the strain of the microbe infecting the subject comprises wild-type nucleotide sequence in a region of the nucleic acid with which mutation is known to be associated with resistance to the antimicrobial agent.

The region may be any length region of nucleic acid of the infecting strain, for example a gene, or a portion of a gene, for example an exon or intron of a gene, or several continuous nucleotides, or a single nucleotide, such as a single nucleotide polymorphism (SNP) associated with resistance to the antimicrobial agent.

In particular, methods of the invention may comprise determining whether nucleic acid of the strain of the microbe infecting the subject comprises wild-type nucleotide sequence at a conserved nucleotide position at which mutation is associated with resistance to the antimicrobial agent in nucleic acid of different resistant strains.

The term ‘conserved nucleotide position’ is used herein to mean that, for a resistant strain, the nucleotide sequence at that position is different from the nucleotide sequence at the corresponding position in a susceptible strain, so the sequence at that nucleotide position is associated with resistance to the antimicrobial agent. In some instances, a conserved nucleotide position may be a single nucleotide polymorphism (SNP), for example a SNP that results in a change in the amino acid sequence encoded by the nucleotide sequence in which the conserved nucleotide position is found (a non-synonymous SNP), or a SNP that does not result in a change in the encoded amino acid sequence (a synonymous SNP). It is also possible that there may be two or more consecutive conserved nucleotide positions associated with resistance to the antimicrobial agent.

In some embodiments, a conserved nucleotide position may be mutated in all known strains of the microbe that are resistant to the antimicrobial agent, and not mutated in all known strains that are susceptible to the antimicrobial agent. If all known strains of the microbe that are resistant to the antimicrobial agent are mutated at the conserved nucleotide position, then determining the presence of a wild-type sequence at that conserved nucleotide position alone will enable a determination that the strain infecting the subject is susceptible to the antimicrobial agent.

For some antimicrobial agents, however, there may not be a conserved nucleotide position that is mutated in all known strains of the microbe that are resistant to that antimicrobial agent. In such circumstances, it may be necessary to determine whether nucleic acid of the strain infecting the subject comprises wild-type nucleotide sequence at a combination of different conserved nucleotide positions, wherein each known resistant strain of the microbe comprises a mutation at one or other of the conserved nucleotide positions of the combination, so that a reliable determination can be made regarding whether the infecting strain is resistant to that antimicrobial agent. For example, a first conserved nucleotide position may be mutated in a first subset of the known strains of the microbe that are resistant to the antimicrobial agent, and a second conserved nucleotide position may be mutated in a different, second subset of the known strains of the microbe that are resistant to the antimicrobial agent. If all the known strains of the microbe that are resistant to the antimicrobial agent are included in the first and the second subsets combined, then determining whether nucleic acid of the strain infecting the subject comprises wild-type nucleotide sequence at the first and second conserved nucleotide positions will be required to determine whether the subject is infected with a strain of the microbe that is susceptible to treatment with the antimicrobial agent.

Alternatively, if there is no conserved nucleotide position that is mutated in all known strains of the microbe that are resistant to the antimicrobial agent, or if there is no combination of conserved nucleotide positions, at least one of which is mutated in each known resistant strain of the microbe, for example only in each of a majority (or in at least 60%, 70%, 80%, or 90%) of the known resistant strains, it can be determined whether it is likely that the strain infecting the subject will be susceptible to the antimicrobial agent by determining whether nucleic acid of the infecting strain comprises wild-type nucleotide sequence at that position, or at that combination of positions.

It can be determined whether a nucleotide position is a conserved nucleotide position by aligning nucleotide sequence of one or more strains of the microbe that are known to be susceptible to the antimicrobial agent with nucleotide sequence of one or more strains of the microbe that are known to be resistant to the antimicrobial agent. Any nucleotide position at which mutations are present in the resistant strains, but not in the susceptible strains, will be a conserved nucleotide position that is associated with resistance. Similar methods can be used to determine whether longer regions of nucleotide sequence are associated with resistance.

Nucleic acid sequence alignment programs are well-known to the skilled person. Examples of suitable programs include multiple sequence alignment programs such as BLAST, Clustal Omega, and Multiple Sequence Comparison by Log-Expectation (MUSCLE).

It can be determined whether a strain of a microbe is susceptible to an antimicrobial agent by exposing a culture of the strain to different dilutions of the antimicrobial agent, for example on agar culture dishes, to determine the minimum concentration of the antimicrobial agent that inhibits growth of the strain (the minimum inhibitory concentration (MIC)). Such techniques are well known to the skilled person. A strain of the microbe that is susceptible to the antimicrobial agent will have a lower MIC than a resistant strain.

Microbes can be categorised into susceptible, intermediately susceptible, and resistant for the relevant antimicrobial agent. The concentration that separates susceptible from non-susceptible microbes is called the S-breakpoint and is expressed as S≤Xmg/L (where X is a MIC value), and the concentration that separates resistant microbes from non-resistant (for example, susceptible or intermediately susceptible) microbes is called the R-breakpoint and is expressed as R>Ymg/L (where Y may be the same or a higher MIC value than X). Clinical breakpoints refer to those MICs that separate strains where there is a high likelihood of treatment success from those where treatment is more likely to fail. In Europe, the European Committee on Antimicrobial Susceptibility Testing (EUCAST), together with the European Medicines Agency (EMA), determines clinical breakpoints for antimicrobial agents (Kahlmeter, Upsala Journal of Medical Sciences, 2014; 119:78-86). These are published, and available on the EUCAST website (www.eucast.org). In the US, breakpoints are determined by the Clinical & Laboratory Standards Institute (CLSI) (www.clsi.org).

It will be appreciated that a ‘wild-type nucleotide sequence’ means a sequence that is present in one or more strains of the microbe that are susceptible to the antimicrobial agent, but not in one or more strains that are resistant to the antimicrobial agent, wherein mutation of the wild-type sequence is associated with resistance to the antimicrobial agent.

The antimicrobial agent may be any antimicrobial agent that prevents or inhibits growth, or replication of a strain of the microbe that is susceptible to the antimicrobial agent, and which may be used for the treatment of an infectious disease caused by the strain in a subject. Examples include an antibiotic, an antiviral agent, or an anti-fungal agent. An antibiotic, for example, may be bacteriostatic or bactericidal.

Examples of infectious diseases caused by microbes for which there are known to exist different strains of the microbe that are resistant to one or more antimicrobial agents are set out in Table 1 below:

TABLE 1
Examples of infectious diseases caused by microbes with antimicrobial resistant strains

| | | Example(s) of antimicrobial |
|---|---|---|
| Disease | Microbe | resistance |
| Urinary tract infections, blood | Escherichia coli | Third generation cephalosporins; |
| stream infections | | fluoroquinolones |
| Pneumonia, blood stream | Klebsiella pneumoniae | Third generation cephalosporins; |
| infections, urinary tract infections | | third generation carbapenems |
| Wound infections, blood stream | Staphylococcus aureus | Methicillin (MRSA) |
| infections | | |
| Pneumonia, meningitis, otitis | Streptococcus pneumoniae | Penicillin |
| Foodborne diarrhoea, blood | Nontyphoidal Salmonella | Fluoroquinolones |
| stream infections | | |
| Diarrhoea (“bacillary dysenteria”) | Shigella species | Fluoroquinolones |
| Gonorrhoea | Neisseria gonorrhoea | Third generation cephalosporins, |
| | | such as cefixime or ceftriaxone; |
| | | fluoroquinolones, such as |
| | | ciprofloxacin; macrolides, such |
| | | as azithromycin; sulfonamides |
| Tuberculosis | Mycobacterium tuberculosis | Isoniazid and rifampin; |
| | | fluoroquinolone; amikacin; |
| | | kanamycin; capreomycin. |
| Malaria | Plasmodium falciparum | Artemisinin-based combination |
| | | therapies (ACTs) |
| Colitis | Clostridium difficile | Metronidazole; vancomycin |
| Acquired immunodeficiency | Human Immunodeficiency | Antiretroviral therapy (ART) |
| syndrome (AIDS) | Virus (HIV) | |
| Influenza | Influenza virus | Adamantanes; neuraminidase |
| | | inhibitors, such as oseltamivir |
| Hepatitis B | Hepatitis B Virus (HBV) | Lamivudine; Adefovir; Entecavir; |
| | | Telbivudine; Tenofovir; |
| | | Emtricitabine |
| Hepatitis C | Hepatitis C virus (HCV) | HCV NS3/4A protease inhibitors: |
| | | telaprevir (Incivek); boceprevir |
| | | (Victrelis) |
| Systemic candidiasis | Candida species | Fluconazole; echinocandins |

Sources include: “Antimicrobial Resistance Global Report on Surveillance” World Health Organization 2014

Resistance determinants and mechanisms in Neisseria gonorrhoeae for antimicrobials previously or currently recommended for treatment of gonorrhoea are described by Unemo and Shafer, Clinical Microbiology Reviews, 2014, Vol. 27(3):587-613, particularly in Table 1 of that document. Known mutations associated with resistance of Neisseria gonorrhoeae to antimicrobial treatment are summarised in Table 2 below.

TABLE 2
Known mutations associated with resistance of
Neisseria gonorrhoeae to antimicrobial treatment

| Antimicrobial agent | Mutation(s) associated with resistance |
|---|---|
| Sulfonamides | Mutations in folP (encoding the sulfonamide target DHPS) |
| | comprise SNPs or a mosaic folP gene containing sequences |
| | from commensal Neisseria spp. |
| Penicillins (e.g., | Mutations in penA (encoding the main lethal target PBP2). |
| penicillin G and | Single amino acid insertion D345 in PBP2 and 4 to 8 |
| ampicillin) | concomitant mutations in the PBP2 carboxyl-terminal region, |
| | decreasing the PBP2 acylation rate and reducing susceptibility |
| | ~6- to 8-fold. More recently, many mosaic penA alleles with up |
| | to 70 amino acid alterations, also reducing PBP2 acylation, |
| | have been described. |
| | Mutations in mtrR, in the promoter (a single nucleotide [A] |
| | deletion in the 13-bp inverted repeat sequence) or coding |
| | sequence (commonly a G45D substitution), result in |
| | overexpression of and increased efflux from the MtrCDE efflux |
| | pump. Rarer mutations resulting in increased MtrCDE efflux are |
| | described in Unemo and Shafer (supra). |
| | porB1b SNPs, for example, encoding G120K and |
| | G120D/A121D mutations in loop 3 of PorB1b, reduce influx |
| | (penB resistance determinants). The penB phenotype is |
| | apparent only in strains with the mtrR resistance determinant. |
| | A SNP in ponA (encoding the second penicillin target, PBP1), |
| | i.e., “ponA1 determinant” (L421P), reduces penicillin acylation |
| | of PBP1 ~2- to 4-fold. |
| Tetracyclines (e.g., | A SNP in rpsJ (encoding ribosomal protein S10), i.e., V57M, |
| tetracycline and | reduces the affinity of tetracycline for the 30S ribosomal target. |
| doxycycline) | mtrR mutations (see above). |
| | penB mutations (see above). |
| | A SNP in pilQ (see above). |
| Spectinomycin | A 16S rRNA SNP, i.e., C1192U, in the spectinomycin-binding |
| | region of helix 34, reduces the affinity of the drug for the |
| | ribosomal target. |
| | Mutations in rpsE (encoding the 30S ribosomal protein S5), i.e., |
| | the T24P mutation and deletions of V25 and K26E, disrupt the |
| | binding of spectinomycin to the ribosomal target. |
| Quinolones (e.g., | gyrA SNPs, e.g., S91F, D95N, and D95G, in the QRDR, reduce |
| ciprofloxacin and | quinolone binding to DNA gyrase. |
| ofloxacin) | parC SNPs, e.g., D86N, S88P, and E91K, in the QRDR, reduce |
| | quinolone binding to topoisomerase IV. |
| | Many additional mutations in the QRDR of gyrA and parC have |
| | been described. An overexpressed NorM efflux pump also |
| | slightly enhances quinolone MICs. |
| Macrolides (e.g., | 23S rRNA SNPs, i.e., C2611T and A2059G (in 1 to 4 alleles), |
| erythromycin and | result in a 23S rRNA target (peptidyltransferase loop of domain |
| azithromycin) | V) with a reduced affinity for the 50S ribosomal macrolide |
| | target. |
| | mtrR mutations (see above). |
| Cephalosporins (e.g., | Mosaic penA alleles encoding mosaic PBP2s with a decreased |
| ceftibuten, cefpodoxime, | PBP2 acylation rate. These proteins have up to 70 amino acid |
| cefixime, cefotaxime, | alterations and are derived from horizontal transfer of partial |
| and ceftriaxone) | penA genes from mainly commensal Neisseria spp. Mutations |
| | in mosaic PBP2s verified to contribute to resistance are A311V, |
| | I312M, V316T, V316P, T483S, A501P, A501V, N512, and |
| | G545S. The resistance mutations need other epistatic |
| | mutations in the mosaic penA allele. |
| | penA SNPs, i.e., A501V and A501T, in nonmosaic alleles can |
| | also enhance cephalosporin MICs. Some additional SNPs |
| | (G542S, P551S, and P551L) were statistically associated with |
| | enhanced cephalosporin MICs, but their effects remain to be |
| | proven with, e.g., site-directed penA mutants in isogenic |
| | backgrounds. |
| | mtrR mutations (see above). |
| | penB mutations (see above). |

The infectious disease may be a sexually transmitted disease. In particular embodiments, the infectious disease is Gonorrhoea. In such embodiments, the antimicrobial agent may be an antibiotic, such as Cephalosporin, Ciprofloxacin, or Azithromycin. The Cephalosporin may be an extended spectrum Cephalosporin (ESC), such as Cefixime or Ceftriaxone.

The EUCAST MIC breakpoints (valid from 1st January 2015) for Neisseria gonorrhoeae are: Cefixime: S≤0.125 mg/L; R>0.125 mg/L; Ceftriaxone: S≤0.125 mg/L; R>0.125 mg/L; Ciprofloxacin: S≤0.03125 mg/L; R>0.0625 mg/L; Azithromycin: S≤0.25 mg/L; R>0.5 mg/L (www.eucastor.org).

In other embodiments, in which the infectious disease is Gonorrhoea, the antimicrobial agent may be a sulphonamide, a penicillin (e.g. penicillin G or ampicillin), a tetracycline (e.g. tetracycline or doxycycline), spectinomycin, a quinolone (e.g. ciproflaxin or ofloxacin), a macrolide (e.g. erythromycin or azithromycin), or a cephalosporin (e.g. ceftibuten, cefpodoxime, cefixime, cefotaxime, or ceftriaxone). In such embodiments, a method of the invention may comprise determining whether nucleic acid of the strain of Neisseria gonorrhoeae infecting the subject comprises wild-type nucleotide sequence of any of the genes recited in Table 2 above, or in any of the regions or conserved nucleotide positions (in particular, SNPs) of the genes recited in Table 2 above, with which mutation is known to be associated with resistance to the antimicrobial agent.

Several mutations in the penA gene (encoding penicillin-binding protein 2, PBP2) have been implicated in ESC resistance in Gonorrhoea, of which the penA mosaic allele is thought to be of significant relevance. Mosaic penA comprises several regions from a number of different Neisseria species, likely acquired by Neisseria gonorrhoeae through genetic transformation. Over 30 mosaic alleles are in circulation, each of which varies in the number and identity of mutations relative to the wild type Gonorrhoea sequence. However, certain mutations are conserved amongst the majority of penA mosaic alleles.

Mosaic penA appears to be the only significant determinant in the development of Cefixime resistance. There is, though, no single mosaic allele that definitively confers resistance. However, the Applicant has appreciated that by identifying patients with wild-type penA sequences, it is possible to identify all patients that are susceptible to Cefixime treatment. Ceftriaxone resistance mechanisms are significantly more complex than those for Cefixime, The presence of any one of the more than 30 penA mosaic alleles is a major factor in the development of resistance, but does not guarantee resistance. Rather, resistance is dependent on a complex synergy of mutations in the penA, mtrR and porB genes. However, all Gonorrhoea strains identified to date with high-level Ceftriaxone resistance have a mosaic penA. Determination of which subjects are infected with strains of N. gonorrhoeae comprising wild-type penA allows the identification of subjects with Gonorrhoea infections that are susceptible to treatment with Ceftriaxone.

Quinolones such as Ciprofloxacin act by inhibiting the activity of two enzymes, DNA gyrase and topoisomerase IV, required for DNA metabolism. Resistance to quinolones developed through the acquisition of single nucleotide polymorphisms (SNPs) in the genes encoding DNA gyrase and topoisomerase IV (gyrA and parC, respectively). Specific SNPs (at S91 and D95) in gyrA alone are sufficient to elicit low- to intermediate-level resistance. High-level resistance requires mutations in both gyrA and parC. Determination of which subjects are infected with strains of N. gonorrhoeae comprising wild-type gyrA allows the identification of subjects with Gonorrhoea that are susceptible to treatment with Ciprofloxacin. This is likely to account for around 50% of subjects suffering from Gonorrhoea, and will enable the use of cheaper antibiotics, whilst preserving use of drugs such as the ESCs as treatment options for as long as possible.

Azithromycin acts by binding to the 23S ribosomal RNA (rRNA), part of the 50S subunit, which leads to inhibition of bacterial protein synthesis. Resistance to Azithromycin can occur by: methylase modification of 23S rRNA; overexpression of efflux pumps, which can act to increase the removal of antibiotics from the cell; or single nucleotide polymorphism (SNP) of particular nucleotides of the 23S rRNA. Azithromycin is the recommended treatment for Chlamydia infection, which is frequently found in Gonorrhoea positive patients. It is administered in conjunction with Ceftriaxone in many developed countries to ensure treatment is successful. Knowing whether subjects are infected with Azithromycin susceptible Gonorrhoea allow it to be used as a monotherapy for those subjects, thus preserving ESCs as a treatment option for other subjects infected with strains of Neisseria gonorrhoeae that are resistant to Azithromycin. Nucleic acid testing is only able to detect resistance that arises as a result of SNPs in the 23S rRNA sequence. However, methylase modifications are very rare in Azithromycin strains. Nucleic acid testing can also be used to detect resistance to Azithromycin that arises as a result of overexpression of efflux pumps.

According to the invention, there is provided a method of determining whether a subject suffering from Gonorrhoea is infected with an antibiotic-susceptible strain of Neisseria gonorrhoeae, which comprises determining whether the strain of Neisseria gonorrhoeae comprises wild-type nucleotide sequence encoding the penA mosaic gene, the gyrA gene, or 23S ribosomal RNA.

According to the invention, there is also provided a method of determining whether a subject suffering from, or suspected of suffering from, Gonorrhoea is infected with a strain of Neisseria gonorrhoeae that is susceptible to an antimicrobial agent, which comprises determining whether nucleic acid of the strain of N. gonorrhoeae infecting the subject comprises wild-type nucleotide sequence at a conserved nucleotide position at which mutation is associated with resistance to the antimicrobial agent in nucleic acid of different strains of N. gonorrhoeae that are resistant to the antimicrobial agent.

According to some embodiments of the invention, mutations at one or more conserved nucleotide positions in the penA mosaic gene are associated with resistance to Cephalosporin. In particular, mutations at nucleotide sequence encoding position F504 and A510 of the penA mosaic gene are conserved in strains that are resistant to Cephalosporin in almost all mosaic alleles, whilst mutations at nucleotide sequence encoding position A501 and A516 of the penA mosaic gene are conserved in strains that are resistant to Cephalosporin in a smaller subset of mosaic alleles.

Thus, in some embodiments of the invention, there is provided a method of determining whether a subject suffering from, or suspected of suffering from, Gonorrhoea is infected with a strain of Neisseria gonorrhoeae that is susceptible to Cephalosporin, which comprises determining whether the strain of N. gonorrhoeae comprises wild-type nucleotide sequence encoding position F504 and/or A510 of the penA mosaic gene. Alternatively, or additionally the method may comprise determining whether the strain of Neisseria gonorrhoeae comprises wild-type nucleotide sequence encoding position A501 and/or A516 of the penA mosaic gene.

In other embodiments, mutation at nucleotide sequence encoding position A501 of a non-mosaic penA gene (in particular, A501V) is conserved in strains that are resistant to Cephalosporin (especially ceftriaxone and cefixime) (Unemo & Shafer, Clinical Microbiology Reviews, 2014, 27(3):587-613). Accordingly, there is also provided according to the invention a method of determining whether a subject suffering from, or suspected of suffering from, Gonorrhoea is infected with a strain of Neisseria gonorrhoeae that is susceptible to Cephalosporin, which comprises determining whether the strain of N. gonorrhoeae comprises wild-type nucleotide sequence encoding position A501 of the penA non-mosaic gene.

According to other embodiments of the invention, mutations at one or more conserved nucleotide positions in the gyrA gene are associated with resistance to Ciprofloxacin. In particular, mutations at nucleotide sequence encoding position S91 and/or D95 of the gyrA gene are conserved in strains that are resistant to Ciprofloxacin.

Thus, in some embodiments of the invention, there is provided a method of determining whether a subject suffering from, or suspected of suffering from, Gonorrhoea is infected with a strain of Neisseria gonorrhoeae that is susceptible to Ciprofloxacin, which comprises determining whether the strain of N. gonorrhoeae comprises wild-type nucleotide sequence encoding position S91 and/or D95 of the gyrA gene.

According to further embodiments of the invention, mutations at one or more conserved nucleotide positions in 23S ribosomal RNA are associated with resistance to Azithromycin. In particular, mutations at nucleotide sequence encoding position 02611 and/or A2059 of 23S ribosomal RNA are conserved in strains that are resistant to Azithromycin.

Specific point mutations of 23S ribosomal RNA can result in varying degrees of resistance. For example, C2611T is associated with strains that have low-level resistance, whereas A2059G is associated with strains that have high-level resistance. The level of resistance to Azithromycin is also linked to the number of mutated 23S alleles. N. gonorrhoeae has four copies of the 23S ribosomal RNA gene. If mutation is observed in only one of the alleles, even if the mutation is A2059G, low levels of resistance will be observed. However, strains with a single mutated allele, while susceptible to treatment with Azithromycin, will quickly develop high-level resistance.

Thus, in some embodiments of the invention, there is provided a method of determining whether a subject suffering from, or suspected of suffering from, Gonorrhoea is infected with a strain of Neisseria gonorrhoeae that is susceptible to Azithromycin, which comprises determining whether the strain of N. gonorrhoeae comprises wild-type nucleotide sequence encoding position C2611 and/or A2059 of 23S ribosomal RNA. Optionally, the method may further comprise determining whether the strain of N. gonorrhoeae does not include mutant nucleotide sequence encoding position C2611 and/or A2059 of 23S ribosomal RNA.

If the strain does not include detectable mutant nucleotide sequence encoding position C2611 and/or A2059 of 23S ribosomal RNA, it can be concluded that all four copies of the 23S ribosomal RNA gene are wild-type, and that the subject is infected with a strain of Neisseria gonorrhoeae that is susceptible to Azithromycin.

It may be determined whether the strain includes mutant nucleotide sequence encoding position C2611 and/or A2059. If any mutant sequence is present (for example, even if only a single copy of the 23S ribosomal RNA gene comprises the mutant sequence) treatment with Azithromycin should be avoided so as not to select for Azithromycin resistance.

Determination of whether the strain of N. gonorrhoeae comprises wild-type nucleotide sequence encoding position C2611 and/or A2059 of 23S ribosomal RNA (and optionally does not include mutant nucleotide sequence encoding position C2611 and/or A2059) may be carried out by detecting for the wild-type (and optionally the mutant) encoding sequence itself, or by detecting for the wild-type (and optionally the mutant) 23S ribosomal RNA sequence encoded by such sequence.

Ng et al. (Antimicrobial Agents and Chemotherapy, 2002, 46(9):3020-3025) describe specific amplification of the four alleles of Neisseria gonorrhoeae 23S ribosomal RNA by PCR using a PCR forward primer of sequence: ACGAATGGCGTAACGATGGCCACA (SEQ ID NO:9) paired with a specific primer for each of the 23S rRNA alleles:

| (SEQ ID NO: 10) | |
|---|---|
| allele 1: | TCAGAATGCCACAGCTTACAAACT; |
| (SEQ ID NO: 11) | |
| allele 2: | GCGACCATACCAAACACCCACAGG; |
| (SEQ ID NO: 12) | |
| allele 3: | GATCCCGTTGCAGTGAAGAAAGTC; |
| (SEQ ID NO: 13) | |
| allele 4: | AACAGACTTACTATCCCATTCAGC |

The allele-specific primers prime downstream of the 23S rRNA.

The PCR conditions used by Ng et al were 1 min of denaturation at 94° C., 1.5 min at 66° C. (for alleles 2 and 3) or 68° C. (for alleles 1 and 4) for annealing, and 2.5 min at 72° C. for elongation for 30 cycles.

The amplicons obtained were then used as templates in a second PCR reaction using a the PCR forward primer of SEQ ID NO:9, and a reverse primer of sequence: TTCGTCCACTCCGGTCCTCTCGTA (SEQ ID NO:14). The conditions for this second PCR reaction were 1 min of denaturation at 94° C., 1 min at 59° C. for annealing, and 1 min at 72° C. for elongation for 35 cycles.

Similar methods may be used according to the invention to determine whether the strain of Neisseria gonorrhoeae infecting the subject comprises wild-type nucleotide sequence encoding position C2611 and/or A2059 of 23S rRNA and, optionally, does not include mutant nucleotide sequence encoding position C2611 and/or A2059 of 23S rRNA. For example, the products of the second PCR reaction may be sequenced, or incubated under hybridizing conditions with a labelled oligonucleotide probe that is able to distinguish between PCR products comprising the wild-type and mutant sequences.

Nucleic acid testing can also be used to detect resistance to Azithromycin that arises as a result of overexpression of efflux pumps. The MtrCDE efflux pump can export structurally diverse hydrophobic antimicrobials. Gonococcal strains showing intermediate-level resistance to substrates of the MtrCDE efflux pump typically have missense mutations in a DNA-binding domain coding region of the mtrR gene (commonly a G45D substitution in the helix-turn-helix domain of amino acid residues 32 to 53), which encodes the MtrR repressor that binds to the mtrCDE promoter. Strains expressing high-level resistance have mutations (most frequently a single ‘A’ nucleotide deletion in a 13-base pair inverted repeat sequence between hexamer sequences at −10 and −35) in the mtrR promoter. Such mutations result in overexpression of, and increased efflux from, the MtrCDE efflux pump (Unemo & Shafer, Clinical Microbiology Reviews, 2014, 27(3):587-613).

Thus, in some embodiments of the invention, a method of determining whether a subject suffering from, or suspected of suffering from, Gonorrhoea is infected with a strain of Neisseria gonorrhoeae that is susceptible to Azithromycin may further comprise determining whether the strain of N. gonorrhoeae comprises a wild-type mtrR promoter sequence (in particular a wild-type 13-base pair repeat sequence between hexamer sequences −10 and −35), and/or a wild-type nucleotide sequence encoding position G45 in the helix-turn-helix domain of amino acid residues 32 to 53 of the mtrR gene.

Zarantonelli et al (Antimicrobial Agents and Chemotherapy, 1999 43(10):2468-2472) and and Ng et al (Antimicrobial Agents and Chemotherapy, 2002, 46(9):3020-3025) describe methods for PCR amplification of the mtrR gene, including the promoter region, using primers: ACTGAAGCTTATTTCCGGCGCAGGCAGGG (SEQ ID NO:15) and GACGACAGTGCCAATGCAACG (SEQ ID NO:16). Such methods may be used according to the invention to determine whether the strain of Neisseria gonorrhoeae infecting the subject comprises wild-type mtrR promoter sequence and/or a wild-type nucleotide sequence encoding position G45 of the mtrR gene. For example, the products of the PCR reaction may be sequenced, or incubated under hybridizing conditions with a labelled oligonucleotide probe that is able to distinguish between PCR products comprising the wild-type and mutant sequences.

Genome sequences for several strains of Neisseria gonorrhoeae have been determined. See, for example: Lewis et at, The Complete Genome Sequence of Neisseria gonorrhoeae (GenBank accession no. AE004969, Neisseria gonorrhoeae FA 1090, complete genome); Chung et al., Complete Genome Sequence of Neisseria gonorrhoeae NCCP11945, Journal of Bacteriology, 2008, 6035-6036 (GenBank accession no. CP001050); Hess et al., Genome Sequence of a Neisseria gonorrhoeae Isolate of a Successful International Clone with Decreased Susceptibility and Resistance to Extended-Spectrum Cephalosporins, Antimicrobial Agents and Chemotherapy, 2012, 56(11):5633-5641 (strain SM-3).

Sequence of the penicillin-binding protein 2 (penA) gene for Neisseria gonorrhoeae strain LM306, based on NCBI GenBank accession number M32091 (version M32091.1; Spratt, Nature (1988) 332 (6160), 173-176), and sequences of the gyrA, and mtrR genes, and the 23S ribosomal RNA alleles, for Neisseria gonorrhoeae strain FA 1090, based on NCBI Reference Sequence NC_002946.2, are provided below. These sequences (or other available Neisseria gonorrhoeae sequences) can be used to design suitable oligonucleotide primers and probes to determine whether a particular wild-type sequence (or combination of wild-type sequences) is present in the strain of Neisseria gonorrhoeae infecting the subject.

If the strain of Neisseria gonorrhoeae comprises wild-type nucleotide sequence encoding the penA mosaic gene, it is expected that the subject can be treated effectively with Cephalosporin as a monotherapy. If the strain of Neisseria gonorrhoeae comprises wild-type nucleotide sequence encoding the gyrA gene, it is expected that the subject can be treated effectively with Ciprofloxacin as a monotherapy. If the strain of Neisseria gonorrhoeae comprises wild-type nucleotide sequence encoding 23S ribosomal RNA (and optionally does not include mutant nucleotide sequence encoding position C2611 and/or A2059 of 23S ribosomal RNA), it is expected that the subject can be treated effectively with Azithromycin as a monotherapy. Similarly, if the strain of Neisseria gonorrhoeae also comprises a wild-type mtrR promoter sequence and/or a wild-type nucleotide sequence encoding position G45 of the mtrR gene, it is expected that the subject can be treated effectively with Azithromycin as a monotherapy.

In some embodiments of the invention, it may be determined whether the subject is infected by a strain of the microbe that is susceptible to any of a plurality of different antimicrobial agents. In such embodiments, the determinations in respect of the different antimicrobial agents may be made at the same time, for example, in a single test. For example, it may be determined whether a subject suffering from, or suspected of suffering from, Gonorrhoea is infected with a strain of Neisseria gonorrhoeae that is susceptible to each of: Ciprofloxacin, Azithromycin, and Cephalosporin; Ciprofloxacin and Azithromycin; Ciprofloxacin and Cephalosporin; or Azithromycin and Cephalosporin.

In other embodiments, it may first be determined for one of the antimicrobial agents whether a subject suffering from, or suspected of suffering from, the infectious disease is infected with a strain of the microbe that is susceptible to that antimicrobial agent. If the subject is found to be infected with a strain of the microbe that is susceptible to the antimicrobial agent, the subject may then be administered with, or prescribed for administration with, the antimicrobial agent as a monotherapy. If the subject is found to be infected with a strain of the microbe that is resistant to the antimicrobial agent, it may then be determined whether the subject is infected with a strain of the microbe that is susceptible to a second antimicrobial agent. If the subject is found to be infected with a strain of the microbe that is susceptible to the second antimicrobial agent, the subject may then be administered with, or prescribed for administration with, the second antimicrobial agent as a monotherapy. If the subject is found to be infected with a strain of the microbe that is resistant to the second antimicrobial agent, and further antimicrobial agents against the microbe are known, it may be determined whether the subject is infected with a strain of the microbe that is susceptible to a third antimicrobial agent, and so on, until an antimicrobial agent is found to which the strain infecting the subject is susceptible. If there is no antimicrobial agent to which the strain infecting the subject is susceptible, the subject may then be administered with, or prescribed for administration with, a combination of two or more of the antimicrobial agents to which the strain is resistant.

For example, in some embodiments of the invention, it may first be determined whether a subject suffering from, or suspected of suffering from, Gonorrhoea is infected with a strain of N. gonorrhoeae that is susceptible to any of the antimicrobial agents selected from Ciprofloxacin, Azithromycin, and Cephalosporin. If it is found that the subject is infected with a strain that is susceptible to the selected antimicrobial agent, the subject can then be administered with that antimicrobial agent as a monotherapy. If it is found that the subject is infected with a strain that is resistant to the selected antimicrobial agent, it may then be determined whether the subject is infected with a strain of N. gonorrhoeae that is susceptible to one of the remaining antimicrobial agents. If it is found that the subject is infected with a strain that is susceptible to the second selected antimicrobial agent, the subject can then be administered with that antimicrobial agent as a monotherapy. If it is found that the subject is infected with a strain that is resistant to the second selected antimicrobial agent, it may then be determined whether the subject is infected with a strain of N. gonorrhoeae that is susceptible to the remaining antimicrobial agent. If it is found that the subject is infected with a strain that is susceptible to the third selected antimicrobial agent, the subject may then be administered with that antimicrobial agent as a monotherapy. If it is found that the subject is infected with a strain that is resistant to the third selected antimicrobial agent, the subject may then be administered with a combination of the first and the second selected antimicrobial agent, the first and the third selected antimicrobial agent, or the second and the third selected antimicrobial agent, or with all three antimicrobial agents.

By such methods, use of antimicrobial agents is limited to treatment of infections caused by strains that are known to be susceptible to the selected antimicrobial agent as a monotherapy, and use of antimicrobial agents against strains that are resistant to the antimicrobial agent is minimised, thereby reducing the amount of selection for such resistant strains. Such methods reduce the prevalence of resistance to antimicrobial agents, as well as the development or spread of resistance, and the development of resistance to multiple antimicrobial agents (multi-drug resistance).

For example, the susceptibility determinations for Ciprofloxacin, Azithromycin, and Cephalosporin may be made in any of the following orders: Cephalosporin, then Azithromycin, then Ciprofloxacin; Cephalosporin, then Ciprofloxacin, then Azithromycin; Azithromycin, then Ciprofloxacin, then Cephalosporin; Azithromycin, then Cephalosporin, then Ciprofloxacin; Ciprofloxacin, then Cephalosporin, then Azithromycin; Ciprofloxacin, then Azithromycin, then Cephalosporin.

For example, in some embodiments of the invention, it may first be determined whether a subject suffering from, or suspected of suffering from, Gonorrhoea is infected with a strain, of N. gonorrhoeae that is susceptible to Ciprofloxacin. If it is found that the subject is infected with a strain that is susceptible to Ciprofloxacin, the subject may then be administered with Ciprofloxacin as a monotherapy. If it is found that the subject is infected with a strain that is resistant to Ciprofloxacin, it may then be determined whether the subject is infected with a strain of N. gonorrhoeae that is susceptible to Azithromycin. If it is found that the subject is infected with a strain that is susceptible to Azithromycin, the subject may then be administered with Azithromycin as a monotherapy. If it is found that the subject is infected with a strain that is resistant to Azithromycin, it may then be determined whether the subject is infected with a strain of N. gonorrhoeae that is susceptible to Cephalosporin. If it is found that the subject is infected with a strain that is susceptible to Cephalosporin, the subject may then be administered with Cephalosporin as a monotherapy. If it is found that the subject is infected with a strain that is resistant to Cephalosporin, the subject may then be administered with Azithromycin and Cephalosporin, or with Ciprofloxacin and Azithromycin, or with Ciprofloxacin and Cephalosporin.

In other embodiments, it may first be determined whether a subject suffering from, or suspected of suffering from, Gonorrhoea is infected with a strain of N. gonorrhoeae that is susceptible to Azithromycin. If it is found that the subject is infected with a strain that is susceptible to Azithromycin, the subject may then be administered with Azithromycin as a monotherapy. If it is found that the subject is infected with a strain that is resistant to Azithromycin, it may then be determined whether the subject is infected with a strain of N. gonorrhoeae that is susceptible to Ciprofloxacin. If it is found that the subject is infected with a strain that is susceptible to Ciprofloxacin, the subject may then be administered with Ciprofloxacin as a monotherapy. If it is found that the subject is infected with a strain that is resistant to Ciprofloxacin, it may then be determined whether the subject is infected with a strain of N. gonorrhoeae that is susceptible to Cephalosporin. If it is found that the subject is infected with a strain that is susceptible to Cephalosporin, the subject may then be administered with Cephalosporin as a monotherapy. If it is found that the subject is infected with a strain that is resistant to Cephalosporin, the subject may then be administered with Azithromycin and Cephalosporin, or with Ciprofloxacin and Azithromycin, or with Ciprofloxacin and Cephalosporin.

Alternatively, susceptibility determinations may be made for two of Ciprofloxacin, Azithromycin, and Cephalosporin in any order, for example Ciprofloxacin then Azithromycin; Ciprofloxacin then Cephalosporin; Azithromycin then Cephalosporin; Azithromycin then Ciprofloxacin; Cephalosporin then Ciprofloxacin; or Ciprofloxacin then Cephalosporin.

There is also provided according to the invention a method for treating a subject infected with N. gonorrhoeae, which comprises:
determining whether the subject is infected with an antibiotic-susceptible strain of N. gonorrhoeae by determining whether the strain of N. gonorrhoeae comprises wild-type nucleotide sequence encoding the penA mosaic gene, the gyrA gene, or 23S ribosomal RNA; and
administering Cephalosporin to the subject as a monotherapy if it is determined that the strain of N. gonorrhoeae comprises wild-type nucleotide sequence encoding the penA mosaic gene; or
administering Ciprofloxacin to the subject as a monotherapy if it is determined that the strain of N. gonorrhoeae comprises wild-type nucleotide sequence encoding the gyrA gene; or
administering Azithromycin to the subject as a monotherapy if it is determined that the strain of N. gonorrhoeae comprises wild-type nucleotide sequence of 23S ribosomal RNA.

It may be determined whether the subject is infected with a Cephalosporin-susceptible strain of N. gonorrhoeae by determining whether the strain of N. gonorrhoeae comprises wild-type nucleotide sequence encoding position F504 and/or A510 of the penA mosaic gene. Optionally, it may be further determined whether the strain of N. gonorrhoeae comprises wild-type nucleotide sequence encoding position A501 and/or A516 of the penA mosaic gene. In other embodiments, it may be determined whether the subject is infected with a Cephalosporin-susceptible strain of N. gonorrhoeae by determining whether the strain of N. gonorrhoeae comprises wild-type nucleotide sequence encoding position A501 of the penA non-mosaic gene.

It may be determined whether the subject is infected with a Ciprofloxacin-susceptible strain of N. gonorrhoeae by determining whether the strain of N. gonorrhoeae comprises wild-type nucleotide sequence encoding position S91 and/or 195 of the gyrA gene.

It may be determined whether the subject is infected with an Azithromycin-susceptible strain of N. gonorrhoeae by determining whether the strain of N. gonorrhoeae comprises nucleic acid with wild-type nucleotide sequence encoding position C2611 and/or A2059 of 23S ribosomal RNA. Optionally, it may also be determined whether the strain of N. gonorrhoeae does not include mutant nucleotide sequence encoding position C2611 and/or A2059 of 23S ribosomal RNA. Optionally, it may be determined whether the strain of N. gonorrhoeae also comprises a wild-type mtrR promoter sequence (in particular, a wild-type 13-base pair repeat sequence between hexamer sequences −10 and −35) and/or a wild-type nucleotide sequence encoding position G45 of the mtrR gene.

Some methods of the invention for treating a subject infected with N. gonorrhoeae comprise:
i) determining whether a subject suffering from, or suspected of suffering from, Gonorrhoea is infected with a strain of N. gonorrhoeae that is susceptible to a first antimicrobial agent;
ii) if it is found that the subject is infected with a strain that is susceptible to the first antimicrobial agent, administering the first antimicrobial agent as a monotherapy to the subject;
iii) if it is found that the subject is infected with a strain that is resistant to the first antimicrobial agent, then determining whether the subject is infected with a strain of N. gonorrhoeae that is susceptible to a second antimicrobial agent;
iv) if it is found that the subject is infected with a strain that is susceptible to the second antimicrobial agent, administering the second antimicrobial agent as a monotherapy to the subject;
v) if it is found that the subject is infected with a strain that is resistant to the second antimicrobial agent, administering the first and the second antimicrobial agents as a combination therapy to the subject.

The first and second antimicrobial agents may be selected from Ciprofloxacin, Azithromycin, and Cephalosporin. For example, the first and second antimicrobial agents may be respectively: Ciprofloxacin and Azithromycin; Ciprofloxacin and Cephalosporin; Azithromycin and Cephalosporin; Azithromycin and Ciprofloxacin; Cephalosporin and Ciprofloxacin; or Ciprofloxacin and Cephalosporin.

In other embodiments, the first and second antimicrobial agents may be selected from any of the antimicrobial agents listed in Table 2 above.

In some embodiments, where there are three antimicrobial agents to test, methods of the invention for treating a subject infected with N. gonorrhoeae may comprise:
i) determining whether a subject suffering from, or suspected of suffering from, an infectious disease is infected with a strain of N. gonorrhoeae that is susceptible to a first antimicrobial agent;
ii) if it is found that the subject is infected with a strain that is susceptible to the first antimicrobial agent, administering the first antimicrobial agent as a monotherapy to the subject;
iii) if it is found that the subject is infected with a strain that is resistant to the first antimicrobial agent, then determining whether the subject is infected with a strain of N. gonorrhoeae that is susceptible to a second antimicrobial agent;
iv) if it is found that the subject is infected with a strain that is susceptible to the second antimicrobial agent, administering the second antimicrobial agent as a monotherapy to the subject;
v) if it is found that the subject is infected with a strain that is resistant to the second antimicrobial agent, then determining whether the subject is infected with a strain of N. gonorrhoeae that is susceptible to a third antimicrobial agent;
vi) if it is found that the subject is infected with a strain that is susceptible to the third antimicrobial agent, administering the third antimicrobial agent as a monotherapy to the subject;
vii) if it is found that the subject is infected with a strain that is resistant to the third antimicrobial agent, administering the first and the second, the first and the third, the second and the third, or the first, second, and the third, antimicrobial agents as a combination therapy to the subject.

For example, the first, second, and third antimicrobial agents may be, respectively: Cephalosporin, Azithromycin, and Ciprofloxacin; Cephalosporin, Ciprofloxacin, and Azithromycin; Azithromycin, Ciprofloxacin, and Cephalosporin; Azithromycin, Cephalosporin, and Ciprofloxacin; Ciprofloxacin, Cephalosporin, and Azithromycin; or Ciprofloxacin, Azithromycin, and Cephalosporin.

In other embodiments, the first, second, and third antimicrobial agents may be selected from any of the antimicrobial agents listed in Table 2 above.

For example, methods of the invention for treating a subject infected with N. gonorrhoeae may comprise:
i) determining whether a subject suffering from, or suspected of suffering from, Gonorrhoea is infected with a strain of N. gonorrhoeae that is susceptible to Ciprofloxacin;
ii) if it is found that the subject is infected with a strain that is susceptible to Ciprofloxacin, administering Ciprofloxacin as a monotherapy to the subject;
iii) if it is found that the subject is infected with a strain that is resistant to Ciprofloxacin, determining whether the subject is infected with a strain of N. gonorrhoeae that is susceptible to Azithromycin;
iv) if it is found that the subject is infected with a strain that is susceptible to Azithromycin, administering Azithromycin as a monotherapy to the subject;
v) if it is found that the subject is infected with a strain that is resistant to Azithromycin, determining whether the subject is infected with a strain of N. gonorrhoeae that is susceptible to Cephalosporin;
vi) if it is found that the subject is infected with a strain that is susceptible to Cephalosporin, administering Cephalosporin as a monotherapy to the subject;
vii) if it is found that the subject is infected with a strain that is resistant to Cephalosporin, administering Azithromycin and Cephalosporin, Ciprofloxacin and Azithromycin, or Ciprofloxacin and Cephalosporin as a combination therapy to the subject.

In an alternative example, methods of the invention for treating a subject infected with N. gonorrhoeae may comprise:
i) determining whether a subject suffering from, or suspected of suffering from, Gonorrhoea is infected with a strain of N. gonorrhoeae that is susceptible to Azithromycin;
ii) if it is found that the subject is infected with a strain that is susceptible to Azithromycin, administering Azithromycin as a monotherapy to the subject;
iii) if it is found that the subject is infected with a strain that is resistant to Azithromycin, determining whether the subject is infected with a strain of N. gonorrhoeae that is susceptible to Ciprofloxacin;
iv) if it is found that the subject is infected with a strain that is susceptible to Ciprofloxacin, administering Ciprofloxacin as a monotherapy to the subject;
v) if it is found that the subject is infected with a strain that is resistant to Ciprofloxacin, determining whether the subject is infected with a strain of N. gonorrhoeae that is susceptible to Cephalosporin;
vi) if it is found that the subject is infected with a strain that is susceptible to Cephalosporin, administering Cephalosporin as a monotherapy to the subject;
vii) if it is found that the subject is infected with a strain that is resistant to Cephalosporin, administering Azithromycin and Cephalosporin, Ciprofloxacin and Azithromycin, or Ciprofloxacin and Cephalosporin as a combination therapy to the subject.

Typically, the subject is a human subject. The subject may be a male or a female human subject. The subject may be symptomatic, or asymptomatic for the infectious disease.

Methods of determining whether nucleic acid of the strain of the microbe infecting the subject comprises wild-type nucleotide sequence may be carried out using nucleic acid obtained from the subject, or using nucleic acid derived from nucleic acid obtained from the subject. Nucleic acid may be derived from nucleic acid obtained from the subject, for example, by nucleic acid amplification of nucleic acid obtained from the subject, or by synthesis of a nucleic acid strand (i.e. sequence) which is complementary to nucleic acid obtained from the subject.

Methods of determining whether nucleic acid of the strain of the microbe infecting the subject comprises wild-type nucleotide sequence may be in vitro methods. The methods may be carried out on a biological sample obtained from the subject. The biological sample may be any biological sample that could contain sufficient quantities of nucleic acid of the strain of the infecting microbe to allow for detection of the wild-type sequence. For example, the biological sample may be a blood, plasma, or a urine sample. Other examples of biological samples include a rectal, oropharyngeal, vaginal, urethral, vulval, meatal, endocervical, serum, skin, or a conjunctival sample. Examples of biological samples to test for presence of N. gonorrhoeae nucleic acid include rectal, oropharyngeal, vaginal, urine, urethral, vulval, meatal, endocervical. Examples of biological samples to test for MRSA include swabs taken from the nostrils, groin, armpit, or skin.

Any suitable method may be used to determine whether the subject is infected with a strain of the microbe comprising nucleic acid that includes the wild-type nucleotide sequence. In some embodiments, it is determined whether the subject is infected with a strain of the microbe comprising nucleic acid that includes the wild-type nucleotide sequence by specifically detecting for the wild-type nucleotide sequence. For example, the wild-type nucleotide sequence may be specifically detected for utilising an oligonucleotide comprising sequence that is complementary to the wild-type nucleotide sequence. Under conditions of suitable stringency, the complementary oligonucleotide will hybridize to nucleic acid comprising the wild-type nucleotide sequence, but not to nucleic acid comprising a mutant sequence. Detecting whether or not the oligonucleotide has hybridized to the nucleic acid can be used to determine whether or not the wild-type nucleotide sequence is present. Suitable techniques for carrying out such methods are well-known to the skilled person.

In other embodiments, the wild-type nucleotide sequence may be detected for utilising an oligonucleotide comprising sequence that is the same sequence as the wild-type nucleotide sequence. Under conditions of suitable stringency, such an oligonucleotide will hybridise to nucleic acid that is complementary to the wild-type nucleotide sequence, but not to nucleic acid that is complementary to a mutant sequence. Detecting whether or not the oligonucleotide has hybridized to the complementary nucleic acid can be used to determine whether or not the wild-type nucleotide sequence is present. Suitable techniques for carrying out such methods are well-known to the skilled person.

Nucleic acid of the strain of the microbe infecting the subject may be present in a biological sample in very low amounts. It may, therefore, be necessary to amplify nucleic acid of the infecting strain to allow a determination of whether or not the wild-type sequence is present. Methods of nucleic acid amplification are well-known to the skilled person. Examples of suitable amplification methods include polymerase chain reaction (PCR), reverse transcription PCR (RT-PCR), isothermal nucleic acid amplification, including transcription-based amplification, such as nucleic acid sequence-based amplification (NASBA), transcription-mediated amplification (TMA), self-sustained sequence replication (3SR) (Chan and Fox, Rev. Med. Microbiol. 10: 185-196 (1999); Guatelli et at, Proc. Natl. Acad. Sci. 87: 1874-1878 (1990); Compton, Nature 350:91-92 (1991)). A further example of a suitable isothermal nucleic acid amplification method is Loop-mediated isothermal amplification (LAMP) (Notomi et al, Nucleic Acids Res. 28 (12): E63).

It will be appreciated that nucleic acid of the strain of the microbe infecting the subject that is used for hybridization to an oligonucleotide that is the same sequence as, or complementary, to the wild-type nucleotide sequence, or that is amplified, to allow a determination of whether or not the wild-type sequence is present, may be microbial genomic nucleic acid, in particular microbial genomic DNA or RNA (for example, genomic RNA of an RNA virus, such as a retrovirus), or may be microbial RNA that has been transcribed from microbial genomic DNA (such as, for example 23S ribosomal RNA).

According to particular embodiments of the invention amplification product may be detected using a dipstick. In suitable methods of dipstick detection, amplification product is transported along a dipstick by capillary action to a capture zone of the dipstick, and detected at the capture zone. Amplification product may be captured and detected using a sandwich nucleic acid dipstick detection assay in which the amplification product is immobilised at the capture zone of the dipstick by hybridisation to a capture probe, and detected at the capture zone by hybridisation to a detection probe.

Methods of detection of nucleic acid by dipstick assay are known to the skilled person. The Applicant has developed particularly sensitive methods of dipstick detection, which are described in WO 02/004667, WO 02/04668, WO 02/004669, WO 02/04671, WO 2008/090340, and in Dineva et al (Journal of Clinical Microbiology, 2005, Vol. 43(8): 4015-4021).

It is well known that a disadvantage of conventional nucleic acid amplification reactions is the risk of contamination of target nucleic acid with non-target nucleic acid that can lead to false positives. Conventionally, the risk of contamination in nucleic acid amplification reactions is minimised by carrying out the reactions in laboratories using separate dedicated areas for sample preparation, nucleic acid amplification, and detection of amplified nucleic acid. It will be appreciated, however, that this is not possible when nucleic acid amplification reactions are carried out away from such facilities (for example in the field, in a physician's office, at home, in remote areas, or in developing countries where specialist facilities may not be available).

The Applicant has appreciated that when a nucleic acid amplification reaction is carried out away from specialised lab facilities, risk of contamination can be reduced by performing the amplification reaction in a processing chamber that is sealed from the external environment. Detection of the amplification product may then be carried out in an analysing chamber that is also sealed from the external environment.

The processing chamber and analysing chamber may be provided by a device. The device may be preloaded with reagents (suitably in lyophilised form) required for amplification of the target nucleic acid (including enzyme activities) and/or detection of the amplification product.

The risk of contamination of other samples with amplification product can be reduced by treatment of the amplification product with nucleic acid modifying or hydrolysing agents that prevent its further amplification. A suitable treatment is chemical treatment that modifies and degrades nucleic acid, for example non-enzymatic degradation of nucleic acid by chemical nucleases. Examples of chemical nucleases are divalent metal chelate complexes, such as copper Phenantroline-Cu (II) or Ascorbate-Cu (II) cleavage, as described by Sigman et al (J. Biol. Chem (1979) 254, 12269-12272) and Chiou (J. Biochem (1984) 96, 1307-1310). Alternatively, a base that is not naturally present in the target nucleic acid can be incorporated into the amplification product. For example, dUTP can be used to incorporate uracil into a DNA amplification product (as described in U.S. Pat. No. 5,035,996). If, prior to amplification, uracil DNA glycosylase (UDG) is then added to a sample that may have been contaminated with such DNA amplification product this will cause enzymatic hydrolysis of any contaminating amplification product (containing uracil) without affecting natural DNA in the sample.

Reagents required for amplification of the target nucleic acid and/or detection of the amplification product may be provided in lyophilised form. Lyophilisation improves the stability of the reagents, thereby allowing them to be stored for longer periods at higher temperatures. Lyophilisation also reduces the weight and volume of the reagents so that they are easier to transport. Use of lyophilised reagents is, therefore, advantageous for carrying out methods of the invention in the field.

The Applicant has developed lyophilisation formulations (i.e. formulations suitable for lyophilisation, described in WO 2008/090340) which (once lyophilised) are able to maintain reagents in a stable condition at temperatures up to 37° C. for at least a year. This removes any requirement for cold storage or cold-chain transport of the reagents. The formulations also have the advantage that they can be rapidly rehydrated after lyophilisation. This is a particularly desirable property of lyophilised formulations used for nucleic acid testing in the field since the speed or accuracy of a test can be adversely affected if a reagent required for amplification of a nucleic acid target or detection of amplification product is not rehydrated readily during the amplification or detection method.

A detection reagent may be used for detection of wild-type nucleotide sequence. The detection reagent may be any suitable reagent for detection of amplification product or a target nucleic acid. The detection reagent may comprise a detection probe that hybridises to the amplification product or target nucleic acid. The detection reagent may itself be labelled (with one or more labels), thereby enabling direct detection of the amplification product or target nucleic acid utilising the detection reagent. Alternatively, a labelling reagent (which comprises one or more labels) for binding the detection reagent may be provided, thereby enabling indirect detection of the amplification product or target nucleic acid utilising the detection and labelling reagents.

The label(s) of the detection reagent (where this is labelled) or labelling reagent may be a visually detectable label. A ‘visually detectable label’ is used herein to include a label that when present in sufficient amounts can be detected by eye, without the aid of instrumentation. Examples of visually detectable labels include colloidal metal sol particles, latex particles, or textile dye particles. An example of colloidal metal sol particles is colloidal gold particles.

The detection reagent may be a detection probe that is provided with a plurality of detection ligands (for example biotin), each of which can be bound by a labelling reagent. Each labelling reagent may comprise a plurality of detection ligand binding moieties, each detection ligand binding moiety being capable of binding a detection ligand of the detection reagent. An example of such a labelling reagent is colloidal gold conjugated to antibiotin antibody. An example of the detection probe and labelling reagent is the detector probe and coloured anti-hapten detection conjugate, respectively, described and illustrated in Dineva et al (Journal of Clinical Microbiology, 2005, Vol. 43(8): 4015-4021).

Detection of the amplification product or target nucleic acid may take place in standard hybridisation buffer. Examples of typical standard hybridisation buffers include a Tris or phosphate buffer comprising salt (suitably 100-400 mM), surfactant (such as PVP), and a detergent.

Preferred methods for amplification and detection of nucleic acid isolated from a biological sample are described in WO 2008/090340.

Examples of suitable nucleic acid amplification primers for generating amplification product, and examples of suitable capture and detection probes, for use in methods of the invention for determining whether a subject suffering from, or suspected of suffering from, Gonorrhoea is infected with a strain of N. gonorrhoeae that is susceptible to an antimicrobial agent include:

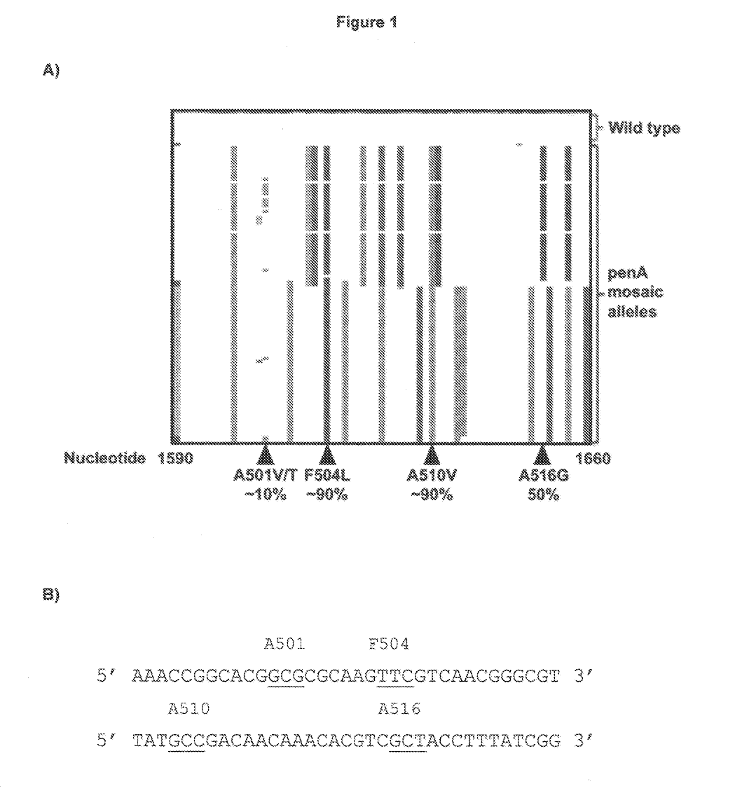

i) a 5′ nucleic acid amplification primer that hybridises under stringent hybridisation conditions upstream of the N. gonorrhoeae nucleic acid sequence shown in FIG. 1; a 3′ nucleic acid amplification primer that hybridises under stringent hybridisation conditions to the opposite strand downstream of the N. gonorrhoeae nucleic acid sequence shown in FIG. 1; and a capture and/or a detection probe that hybridises under stringent hybridisation conditions to a region of N. gonorrhoeae nucleic acid that encodes position F504 and A510 of the penA mosaic gene, wherein the capture and/or detection probe comprises nucleotide sequence that is complementary to, or the same sequence as, wild-type nucleotide sequence encoding position F504 and A510 of the penA mosaic gene;
ii) a 5′ nucleic acid amplification primer that hybridises under stringent hybridisation conditions to a region of N. gonorrhoeae nucleic acid that encodes position F504 of the penA mosaic gene wherein the 5′ primer comprises nucleotide sequence that is complementary to, or the same sequence as, wild-type nucleotide sequence encoding position F504 of the penA mosaic gene; a 3′ nucleic acid amplification primer that hybridises under stringent hybridisation conditions to the opposite strand downstream of the N. gonorrhoeae nucleic acid sequence shown in FIG. 1; a capture and/or detection probe that hybridises under stringent hybridisation conditions to a region of N. gonorrhoeae nucleic acid that encodes position A510 of the penA mosaic gene, wherein the capture and/or detection probe comprises nucleotide sequence that is complementary to, or the same sequence as, wild-type nucleotide sequence encoding position A510 of the penA mosaic gene;
iii) a 5′ nucleic acid amplification primer that hybridises under stringent hybridisation conditions upstream of the N. gonorrhoeae nucleic acid sequence shown in FIG. 1; a 3′ nucleic acid amplification primer that hybridises under stringent hybridisation conditions to the opposite strand to a region of N. gonorrhoeae nucleic acid that encodes position A510 of the penA mosaic gene, wherein the 3′ primer comprises nucleotide sequence that is complementary to, or the same sequence as, wild-type nucleotide sequence encoding position A510 of the penA mosaic gene; a capture and/or detection probe that hybridises under stringent hybridisation conditions to a region of N. gonorrhoeae nucleic acid that encodes position F504 of the penA mosaic gene, wherein the capture and/or detection probe comprises nucleotide sequence that is complementary to, or the same sequence as, wild-type nucleotide sequence encoding position F504 of the penA mosaic gene.

There is also provided according to the invention a kit for determining whether a subject suffering from Gonorrhoea is infected with an antibiotic-susceptible strain of Neisseria gonorrhoeae, which comprises:

i) an oligonucleotide that hybridizes under stringent conditions to N. gonorrhoeae nucleic acid comprising sequence that is the same sequence as, or complementary to, wild-type nucleotide sequence of the penA mosaic gene, wherein the oligonucleotide comprises nucleotide sequence that is complementary to, or the same sequence as, wild-type nucleotide sequence encoding position F504 and/or A510 of the penA mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position F504 and/or A510 of the penA mosaic gene; or
ii) an oligonucleotide that hybridizes under stringent conditions to N. gonorrhoeae nucleic acid comprising sequence that is the same sequence as, or complementary to, wild-type nucleotide sequence of the penA mosaic gene, wherein the oligonucleotide comprises nucleotide sequence that is complementary to, or the same sequence as, wild-type nucleotide sequence encoding position A501 and/or A516 of the penA mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position A501 and/or A516 of the penA mosaic gene; or
iii) an oligonucleotide that hybridizes under stringent conditions to N. gonorrhoeae nucleic acid comprising sequence that is the same sequence as, or complementary to, wild-type nucleotide sequence of the gyrA gene, wherein the oligonucleotide comprises nucleotide sequence that is complementary to, or the same sequence as, wild-type nucleotide sequence encoding position S91 and/or D95 of the gyrA gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position S91 and/or D95 of the gyrA gene; or
iv) an oligonucleotide that hybridizes under stringent conditions to N. gonorrhoeae nucleic acid comprising sequence that is the same sequence as, or complementary to, wild-type nucleotide sequence of 23S ribosomal RNA, wherein the oligonucleotide comprises nucleotide sequence that is complementary to, or the same sequence as, wild-type nucleotide sequence encoding position C2611 and/or A2059 of 23S ribosomal RNA, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position C2611 and/or A2059 of 23S ribosomal RNA.

There is also provided according to the invention a kit for determining whether a subject suffering from Gonorrhoea is infected with an antibiotic-susceptible strain of Neisseria gonorrhoeae, which comprises the oligonucleotide of (i) and/or (ii) and/or (iii) and/or (iv) above, and/or:

(v) an oligonucleotide that hybridizes under stringent conditions to N. gonorrhoeae nucleic acid comprising sequence that is the same sequence as, or complementary to, wild-type nucleotide sequence of the penA non-mosaic gene, wherein the oligonucleotide comprises nucleotide sequence that is complementary to, or the same sequence as, wild-type nucleotide sequence encoding position A501 of the penA non-mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position A501 of the penA non-mosaic gene.

A kit of the invention which comprises the oligonucleotide of (iv) above may further comprise:

vi) an oligonucleotide that hybridizes under stringent conditions to N. gonorrhoeae nucleic acid comprising sequence that is the same sequence as, or complementary to, wild-type nucleotide sequence from position −10 to −35 of the mtrR promoter, wherein the oligonucleotide comprises nucleotide sequence that is complementary to, or the same sequence as, wild-type nucleotide sequence from −10 to −35 of the mtrR promoter, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae comprising a mutation at a position from −10 to −35 of the mtrR promoter; and/or
vii) an oligonucleotide that hybridizes under stringent conditions to N. gonorrhoeae nucleic acid comprising sequence that is the same sequence as, or complementary to, wild-type nucleotide sequence encoding position G45 of the mtrR gene, wherein the oligonucleotide comprises nucleotide sequence that is complementary to, or the same sequence as, wild-type nucleotide sequence encoding position G45 of the mtrR gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position G45 of the mtrR gene.

The oligonucleotides may be selected from oligonucleotides that hybridize under stringent conditions to nucleic acid comprising sequence that is the same sequence as, or complementary to, the nucleotide sequence of:

(SEQ ID NO: 1)
AAACCGGCACGGCGCGCAAGTTCGTCAACGGGCGT;
(SEQ ID NO: 2)
TATGCCGACAACAAACACGTCGCTACCTTTATCGG;
(SEQ ID NO: 3)
AAATACCACCCCCACGGCGATTCCGCAGTTTACGAC;
(SEQ ID NO: 4)
ACCATCGTCCGTATGGCGCAAAATTTCGCTATGCGT;
(SEQ ID NO: 5)
GAAGATGCAATCTACCCGCTGCTAGACGGAAAGACCCCGTGAACCTTTAC
TGTAGCTTTGC;
or
(SEQ ID NO: 6)
CATTTAAAGTGGTACGTGAGCTGGGTTTAAAACGTCGTGAGACAGTTTGG
TCCCTATCTGCAGTGGG
or;
(SEQ ID NO: 7)
AAACCGGCACGGCGCGCAAGTTCGTCAACGGGCGTTATGCCGACAACAAA
CACGTCGCTACCTTTATCGG;
or
(SEQ ID NO: 8)
AAATACCACCCCCACGGCGATTCCGCAGTTTACGACACCATCGTCCGTAT
GGCGCAAAATTTCGCTATGCGT.

The oligonucleotide may be at least 10, 15, or 20 nucleotides in length. The oligonucleotide may be upto 30, 40, 50, or 100 nucleotides in length.

The oligonucleotide may be at least 25, 30, 35, 40, 45, 50, or over 50 nucleotides in length, for example over 50 to 100 nucleotides in length.

The oligonucleotide may comprise nucleotide sequence that is at least 90%, 91%, 92%, 93%, 94%, 95%, 96%, 97%, 98%, or 99% identical, or that is 100% identical, to the nucleotide sequence of any of SEQ ID NOs: 1-8, or the complement thereof.

The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]+0.41x %[G/C^{b}]−500x[L^{c}]⁻¹−0.61x % formamide;

2) DNA-RNA or RNA-RNA hybrids

Tₘ=79.8° C.+18.5(log₁₀[Na⁺]^{a})+0.58(% G/C^{b})+11.8(% G/C^{b})²-820/L^{c};

3) oligo-DNA or oligo-RNAs hybrids:
For <20 nucleotides: Tₘ=2(lₙ);
For 20-35 nucleotides: Tₘ=221-1.46(lₙ);
   ^{a} or for other monovalent cation, but only accurate in the 0.01-0.4 M range,
   ^{b} only accurate for % GC in the 30% to 75% range.
   ^{c} L=length of duplex in base pairs.
   ^{d} oligo, oligonucleotide; 1ₙ=effective length of primer=2x (no. of G/C)+(no. of NT).

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical stringent conditions (also referred to as high stringency hybridisation conditions) for DNA hybrids longer than 50 nucleotides encompass hybridisation at 65° C. in 1×SSC or at 42° C. in 1×SSC and 50% formamide, followed by washing at 65° C. in 0.3×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3rd Edition, Cold Spring Harbor Laboratory Press, CSH, NewYork or to Current Protocols in Molecular Biology, John Wiley & Sons, N.Y. (1989 and yearly updates). The oligonucleotide may be labelled, for example with a visually detectable label. Examples of visually detectable labels include colloidal metal sol particles, latex particles, or textile dye particles. An example of colloidal metal sol particles is colloidal gold particles.

The kit of the invention may comprise any combination of oligonucleotides (i), (ii), (iii), and (iv) above, for example (i)+(ii), (ii)+(iii), (iii)+(iv), (i)+(iii), (i)+(iv), (ii)+(iv), or (i)+(ii)+(iii), (i)+(ii)+(iv), (i)+(iii)+(iv), (ii)+(iii)+(iv), or (i)+(ii)+(iii)+(iv). If oligonucleotide (ii) is present, it is preferred that oligonucleotide (i) is also present.

In other embodiments, a kit of the invention may comprise any combination of oligonucleotides (i)-(vii) above, for example:
(i) (+optionally (ii))+(iii);
(i) (+optionally (ii))+(iv) (+optionally (vi)+/or (vii);
(i) (+optionally (ii))+(iii)+(iv) (+optionally (vi)+/or (vii);
(v)+(iii);
(v)+(iv) (+optionally (vi)+/or (vii);
(v)+(iii)+(iv) (+optionally (vi)+/or (vii);
(iii)+(iv) (+optionally (vi)+/or (vii);
(iii)+(v);
(iv)+(vi)+/or (vii).

A kit of the invention may further comprise oligonucleotide primers for amplification of N. gonorrhoeae nucleic acid that comprises the wild-type nucleotide sequence encoding position: A501 and/or A516 of the penA mosaic gene; S91 and/or D95 of the gyrA gene; or 02611 and/or A2059 of 23S ribosomal RNA.

A kit of the invention may further comprise oligonucleotide primers for amplification of Neisseria gonorrhoeae nucleic acid that comprises the wild-type nucleotide sequence encoding position: F504 and/or A510 of the penA mosaic gene and optionally, A501 and/or A516 of the penA mosaic gene; S91 and/or D95 of the gyrA gene; or C2611 and/or A2059 of 23S ribosomal RNA.

A kit of the invention may further comprise oligonucleotide primers for amplification of Neisseria gonorrhoeae nucleic acid that comprises the wild-type nucleotide sequence encoding position: F504 and/or A510 of the penA mosaic gene and optionally, A501 and/or A516 of the penA mosaic gene; A501 of the penA non-mosaic gene; S91 and/or D95 of the gyrA gene; C2611 and/or A2059 of 23S ribosomal RNA and, optionally, −10 to −35 of the mtrR promoter and/or G45 of the mtrR gene.

Sequence of the penicillin-binding protein 2 (penA) gene for Neisseria gonorrhoeae strain LM306, based on NCBI GenBank accession number M32091 (version M32091.1; Spratt, Nature (1988) 332 (6160), 173-176) is provided below, as well as the amino acid sequence encoded by the gene. Conserved nucleotide positions, mutation of which is associated with antimicrobial resistance, and their corresponding encoded amino acid sequence is shown underlined in bold, and highlighted.

```
Penicillin-binding protein 2 (penA) gene: nucleotide sequence M32091;
(NCBI Accession Version M32091.1; GI 150278)
(SEQ ID NO: 17)
      1 atgttgattaaaagcgaatataagccccggatgctgcccaaagaagagcaggtcaaaaag
    61 ccgatgaccagtaacggacggattagcttcgtcctgatggcaatggcggtcttgtttgcc
  121 tgtctgattgcccgcgggctgtatctgcagacggtaacgtataactttttgaaagaacag
  181 ggcgacaaccggattgtgcggactcaagcattgccggctacacgcggtacggtttcggac
  241 cggaacggtgcggttttggcgttgagcgcgccgacggagtccctgtttgccgtgcctaaa
  301 gatatgaaggaaatgccgtctgccgcccaattggaacgcctgtccgagcttgtcgatgtg
  361 ccggtcgatgttttgaggaacaaactcgaacagaaaggcaagtcgtttatttggatcaag
  421 cggcagctcgatcccaaggttgccgaagaggtcaaagccttgggtttggaaaactttgta
  481 tttgaaaaagaattaaaacgccattacccgatgggcaacctgtttgcacacgtcatcgga
  541 tttaccgatattgacggcaaaggtcaggaaggtttggaactttcgcttgaagacagcctg
  601 tatggcgaagacggcgcggaagttgttttgcgggaccggcagggcaatattgtggacagc
  661 ttggactccccgcgcaataaagcaccgcaaaacggcaaagacatcatcctttccctcgat
  721 cagaggattcagaccttggcctatgaagagttgaacaaggcggtcgaataccatcaggca
  781 aaagccggaacggtggtggttttggatgcccgcacgggggaaatcctcgccttggccaat
  841 acgcccgcctacgatcccaacagacccggccgggcagacagcgaacagcggcgcaaccgt
  901 gccgtaaccgatatgatcgaacctggttcggcaatcaaaccgttcgtgattgcgaaggca
  961 ttggatgcgggcaaaaccgatttgaacgaacggctgaatacgcagccttataaaatcgga
1021 ccgtctcccgtgcgcgatacccatgtttacccctctttggatgtgcgcggcattatgcag
1081 aaatcgtccaacgtcggcacaagcaaactgtctgcgcgtttcggcgccgaagaaatgtat
1141 gacttctatcatgaattgggcatcggtgtgcgtatgcactcgggctttccgggggaaact
1201 gcaggtttgttgagaaattggcgcaggtggcggcccatcgaacaggcgacgatgtctttc
1261 ggttacggtctgcaattgagcctgctgcaattggcgcgcgcctataccgcactgacgcac
1321 gacggcgttttgctgccgctcagctttgagaagcaggcggttgcgccgcaaggcaaacgc
1381 atattcaaagaatcgaccgcgcgcgaggtacgcaatctgatggtttccgtaaccgagccg
1441 ggcggcaccggtacggcgggtgcggtggacggtttcgatgtcggcgctaaaaccggcacg

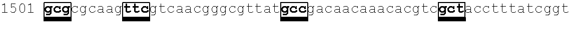

1561 tttgcccccgccaaaaacccccgtgtgattgtggcggtaaccatcgacgaaccgactgcc
1621 cacggctattacggcggcgtagtggcagggccgcccttcaaaaaaattatgggcggcagc
1681 ctgaacatcttgggcatttccccgaccaagccactgaccgccgcagccgtcaaaacaccg
1741 tcttaa
Penicillin-binding protein 2 (penA) gene: protein sequence (NCBI
Accession M32091; Version M32091.1; GI 150278; protein id AAA25463.1)
(SEQ ID NO: 18)
      1 MLIKSEYKPR MLPKEEQVKK PMTSNGRISF VLMAMAVLFA CLIARGLYLQ TVTYNFLKEQ
    61 GDNRIVRTQA LPATRGTVSD RNGAVLALSA PTESLFAVPK DMKEMPSAAQ LERLSELVDV
  121 PVDVLRNKLE QKGKSFIWIK RQLDPKVAEE VKALGLENFV FEKELKRHYP MGNLFAHVIG
  181 FTDIDGKGQE GLELSLEDSL YGEDGAEVVL RDRQGNIVDS LDSPRNKAPQ NGKDIILSLD
  241 QRIQTLAYEE LNKAVEYHQA KAGTVVVLDA RTGEILALAN TPAYDPNRPG RADSEQRRNR
  301 AVTDMIEPGS AIKPFVIAKA LDAGKTDLNE RLNTQPYKIG PSPVRDTHVY PSLDVRGIMQ
  361 KSSNVGTSKL SARFGAEEMY DFYHELGIGV RMHSGFPGET AGLLRNWRRW RPIEQATMSF
  421 GYGLQLSLLQ LARAYTALTH DGVLLPLSFE KQAVAPQGKR IFKESTAREV RNLMVSVTEP

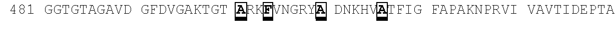

  541 HGYYGGVVAG PPFKKIMGGS LNILGISPTK PLTAAAVKTP S
```

Sequences of the gyrA, and mtrR genes, and the 23S ribosomal RNA alleles, for Neisseria gonorrhoeae strain FA 1090, based on NCBI Reference Sequence NC_002946.2 (locus NC_002946; GenBank: AE004969.1) are provided below, as well as the amino acid sequences encoded by the gyrA, and mtrR genes. Conserved nucleotide positions, mutation of which is associated with antimicrobial resistance, and their corresponding encoded amino acid sequence (where appropriate) is shown underlined in bold, and highlighted.

```
gyrA gene: nucleotide sequence (NCBI GeneID 3282891; Gene symbol
NGO0629)
(SEQ ID NO: 19)
      1 atgaccgacg caaccatccg ccacgaccac aaattcgccc tcgaaaccct gcccgtcagc
    61 cttgaagacg aaatgcgcaa aagctatctc gactacgcca tgagcgtcat tgtcgggcgc
  121 gcgctgccgg acgttcgcga cggcctaaag ccggtgcacc ggcgcgtact gtacgcgatg
  181 cacgagctga aaaataactg gaatgccgcc tacaaaaaat cggcgcgcat cgtcggcgac

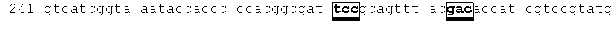

  301 gcgcaaaatt tcgctatgcg ttatgtgctg atagacggac agggcaactt cggatcggtg
  361 gacgggcttg ccgccgcagc catgcgctat accgaaatcc gcatggcgaa aatctcacat
  421 gaaatgctgg cagacattga ggaagaaacc gttaatttcg gcccgaacta cgacggtagc
  481 gaacacgagc cgcttgtact gccgacccgt ttccccacac tgctcgtcaa cggctcgtcc
  541 ggtatcgccg tcggtatggc gaccaacatc ccgccgcaca acctcaccga caccatcaac
  601 gcctgtctgc gtcttttgga cgaacccaaa accgaaatcg acgaactgat cgacattatc
  661 caagcccccg acttcccgac cggggcaacc atctacggct tgggcggcgt gcgcgaaggc
  721 tataaaacag gccgcggccg cgtcgttata cgcggtaaga cccatatcga acccataggc
  781 aaaaacggcg aacgcgaagc catcgttatc gacgaaatcc cctatcaggt caacaaagcc
  841 aagttggtcg agaaaatcgg cgatttggtt cgggaaaaaa cgctggaagg catttccgag
  901 ctccgcgacg aatccgacaa atccgggatg cgcgtcgtta tcgagctgaa acgcaacgaa
  961 aatgccgaag tcgtcttaaa ccaactctac aaactgactc cgctgcaaga cagtttcggc
1021 atcaatatgg ttgttttggt cgacggacaa ccgcgcctgt taaacctgaa acagattctc
1081 tccgaattcc tgcgccaccg ccgcgaagtc gttacccgac gtacgctttt ccggctgaag
1141 aaggcacgcc atgaagggca tatcgccgaa ggcaaagccg tcgcactgtc caatatcgat
1201 gaaatcatca agctcatcaa agaatcgccc aacgcggccg aggccaaaga aaaactgctt
1261 gcgcgccctt ggcgcagcag cctcgttgaa gaaatgctga cgcgttccgg tctggatttg
1321 gaaatgatgc gtccggaagg attggctgca aacattggtc tgaaaaaaca aggttattac
1381 ctgagcgaga ttcaggcaga tgctatttta cgcatgagcc tgcgaaacct gaccggcctc
1441 gatcagaaag aaattatcga aagctacaaa aacctgatgg gtaaaatcat cgactttgtg
1501 gatatcctct ccaaacccga acgcattacc caaatcatcc gtgacgaact ggaagaaatc
1561 aaaaccaact atggcgacga acgccgcagc gaaatcaacc cgttcggcgg cgacattgcc
1621 gatgaagacc tgattccgca acgcgaaatg gtcgtgaccc tgacccacgg cggctatata
1681 aaaacccagc cgaccaccga ctatcaggct cagcgtcgcg gcgggcgcgg caaacaggcg
1741 gctgccacca aagacgaaga ctttatcgaa accctgtttg ttgccaacac gcatgactat
1801 ttgatgtgtt ttaccaacct cggcaagtgc cactggatta aggtttacaa actgcccgaa
1861 ggcggacgca acagccgcgg ccgtccgatt aacaacgtca tccagctgga agaaggcgaa
1921 aaagtcagcg cgattctggc agtacgcgag tttcccgaag accaatacgt cttcttcgcc
1981 accgcgcagg gaatggtgaa aaaagtccaa ctttccgcct ttaaaaacgt ccgcgcccaa
2041 ggcattaaag ccatcgcact caaagaaggc gactacctcg tcggcgctgc gcaaacaggc
2101 ggtgcggacg acattatgtt gttctccaac ttgggcaaag ccatccgctt caacgaatac
2161 tgggaaaaat ccggcaacga cgaagcggaa gatgccgaca tcgaaaccga gatttcagac
2221 gacctcgaag acgaaaccgc cgacaacgaa aacaccctgc caagcggcaa aaacggcgtg
2281 cgtccgtccg gtcgcggcag cggcggtttg cgcggtatgc gcctgcctgc cgacggcaaa
2341 atcgtcagcc tgattacctt cgcccctgaa accgaagaaa gcggtttgca agttttaacc
2401 gccaccgcca acggatacgg aaaacgcacc ccgattgccg attacagccg caaaaacaaa
2461 ggcgggcaag gcagtattgc cattaacacc ggcgagcgca acggcgattt ggtcgccgca
2521 accttggtcg gcgaaaccga cgatttgatg ctgattacca gcggcggcgt gcttatccgt
2581 accaaagtcg aacaaatccg cgaaaccggc cgcgccgcag caggcgtgaa actgattaac
2641 ttggacgaag gcgaaacctt ggtatcgctg gaacgtgttg ccgaagacga atccgaactc
2701 tccggcgctt ctgtaatttc caatgtaacc gaaccggaag ccgagaactg a
gyrA gene: protein sequence (GenBank accession no. AAW89357)
(SEQ ID NO: 20)
      1 MTDATIRHDH KFALETLPVS LEDEMRKSYL DYAMSVIVGR ALPDVRDGLK PVHRRVLYAM

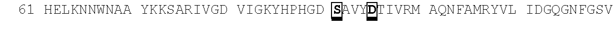

  121 DGLAAAAMRY TEIRMAKISH EMLADIEEET VNFGPNYDGS EHEPLVLPTR FPTLLVNGSS
  181 GIAVGMATNI PPHNLTDTIN ACLRLLDEPK TEIDELIDII QAPDFPTGAT IYGLGGVREG
  241 YKTGRGRVVI RGKTHIEPIG KNGEREAIVI DEIPYQVNKA KLVEKIGDLV REKTLEGISE
  301 LRDESDKSGM RVVIELKRNE NAEVVLNQLY KLTPLQDSFG INMVVLVDGQ PRLLNLKQIL
  361 SEFLRHRREV VTRRTLFRLK KARHEGHIAE GKAVALSNID EIIKLIKESP NAAEAKEKLL
  421 ARPWRSSLVE EMLTRSGLDL EMMRPEGLAA NIGLKKQGYY LSEIQADAIL RMSLRNLTGL
  481 DQKEIIESYK NLMGKIIDFV DILSKPERIT QIIRDELEEI KTNYGDERRS EINPFGGDIA
  541 DEDLIPQREM VVTLTHGGYI KTQPTTDYQA QRRGGRGKQA AATKDEDFIE TLFVANTHDY
  601 LMCFTNLGKC HWIKVYKLPE GGRNSRGRPI NNVIQLEEGE KVSAILAVRE FPEDQYVFFA
  661 TAQGMVKKVQ LSAFKNVRAQ GIKAIALKEG DYLVGAAQTG GADDIMLFSN LGKAIRFNEY
  721 WEKSGNDEAE DADIETEISD DLEDETADNE NTLPSGKNGV RPSGRGSGGL RGMRLPADGK
  781 IVSLITFAPE TEESGLQVLT ATANGYGKRT PIADYSRKNK GGQGSIAINT GERNGDLVAA
  841 TLVGETDDLM LITSGGVLIR TKVEQIRETG RAAAGVKLIN LDEGETLVSL ERVAEDESEL
  901 SGASVISNVT EPEAEN
23S rRNA allele 1 nucleotide sequence (NCBI GeneID: 3370843; Gene
symbol: NGO r02)
(SEQ ID NO: 21)
      1 tgaaatgata gagtcaagtg aataagtgca tcaggcggat gccttggcga tgataggcga
    61 cgaaggacgt gtaagcctgc gaaaagcgcg ggggagctgg caataaagca atgatcccgc
  121 ggtgtccgaa tggggaaacc cactgcattc tgtgcagtat cctaagttga atacataggc
  181 ttagagaagc gaacccggag aactgaacca tctaagtacc cggaggaaaa gaaatcaacc
  241 gagattccgc aagtagtggc gagcgaacgc ggaggagcct gtacgtaata actgtcgagg
  301 tagaagaaca agctgggaag cttgaccata gcgggtgaca gtcccgtatt cgaaatctca
  361 acagcggtac taagcgtacg aaaagtaggg cgggacacgt gaaatcctgt ctgaatatgg
  421 ggggaccatc ctccaaggct aaatactcat catcgaccga tagtgaacca gtaccgtgag
  481 ggaaaggcga aaagaacccc gggaggggag tgaaacagaa cctgaaacct gatgcataca
  541 aacagtggga gcgccctagt ggtgtgactg cgtacctttt gtataatggg tcaacgactt
  601 acattcagta gcgagcttaa ccggataggg gaggcgtagg gaaaccgagt cttaataggg
  661 cgatgagttg ctgggtgtag acccgaaacc gagtgatcta tccatggcca ggttgaaggt
  721 gccgtaacag gtactggagg accgaaccca cgcatgttgc aaaatgcggg gatgagctgt
  781 gggtaggggt gaaaggctaa acaaactcgg agatagctgg ttctccccga aaactattta
  841 ggtagtgcct cgagcaagac actgatgggg gtaaagcact gttatggcta gggggttatt
  901 gcaacttacc aacccatggc aaactcagaa taccatcaag tggttcctcg ggagacagac
  961 agcgggtgct aacgtccgtt gtcaagaggg aaacaaccca gaccgccggc taaggtccca
1021 aatgatagat taagtggtaa acgaagtggg aaggcacaga cagccaggat gttggcttag
1081 aagcagccat catttaaaga aagcgtaata gctcactggt cgagtcgtcc tgcgcggaag
1141 atgtaacggg gctcaaatct ataaccgaag ctgcggatgc cggtttaccg gcatggtagg
1201 ggagcgttct gtaggctgat gaaggtgcat tgtaaagtgt gctggaggta tcagaagtgc
1261 gaatgttgac atgagtagcg ataaagcggg tgaaaagccc gctcgccgaa agcccaaggt
1321 ttcctacgca acgttca:cg gcgtagggtg agtcggcccc taaggcgagg cagaaatgcg
1381 tagtcgatgg gaaacaggtt aatattcctg tacttgattc aaatgcgatg tggggacgga
1441 gaaggttagg ttggcaagct gttggaatag cttgtttaag ccggtaggtg gaagacttag
1501 gcaaatccgg gttttcttaa caccgaagaa gtgatgacga gtgtttacgg acacgaagca
1561 accgatacca cgcttccagg aaaagccact aagcttcagt ttgaatcgaa ccgtaccgca
1621 aaccgacaca ggtgggcagg atgagaattc taaggcgctt gagagaactc gggagaagga
1681 actcggcaaa ttgataccgt aacttcggga gaaggtatgc cctctaaggt taaggacttg
1741 ctccgtaagc cccggagggt cgcagagaat aggtggctgc gacttgttta ttaaaaacac
1801 gagcactctt gccaacacga aagtggacgt atagggtgta acgcctgccc ggtgccggaa
1861 ggttaattga agatgtgcaa gcatcggatc gaagccccgg taaacggcgg ccgtaactat
1921 aacggtccta aggtagcgaa attccttgtc gggtaagttc cgacccgcac gaatggcgta
1981 acgatggcca cactgtctcc tcccgagact cagcgaagtt gaagtggttg tgaagatgca

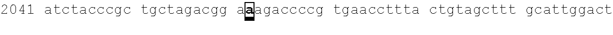

2101 ttgaagtcac ttgtgtagga taggtggaag gcttggaagc aaagacgcca gtctctgtgg
2161 agtcgtcctt gaaaatacca ccctggtgtc tttgaggttc taacccagac ccgtcatccg
2221 ggtcggggac cgtgcatggt aggcagtttg actggggcgg tctcctccca aagcgtaacg
2281 gaggagttcg aaggttacct aggtccggtc ggaaatcgga ctgatagtgc aatggcaaaa
2341 ggtagcttaa ctgcgagacc gacaagtcgg gcaggtgcga aagcaggaca tagtgatccg
2401 gtggttctgt atggaagggc catcgctcaa cggataaaag gtactccggg gataacaggc
2461 ttgattccgc ccaagagttc atatcgacgg cggagtttgg cacctcgatg tcggctcatc
2521 acatcctggg gctgtagtcg gtcccaaggg tatggctgtt cgccatttta aagtggtacg

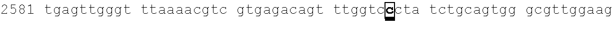

2641 tttgacgggg gctgctccta gtacgagagg accggagtgg acgaacctct ggtgtaccgg
2701 ttgtaacgcc agttgcatag ccgggtagct aagttcggaa gagataagcg ctgaaagcat
2761 ctaagcgcga aactcgcctg aagatgagac ttcccttgcg gtttaaccgc actaaagggt
2821 cgttcgagac caggacgttg ataggtgggg tgtggaagcg cggtaacgcg tgaagctaac
2881 ccatactaat tgcccgtgag gcttgactct
23S rRNA allele 2 nucleotide sequence (NCBI GeneID: 3370844; Gene
symbol: NGO r05)
(SEQ ID NO: 22)
      1 tgaaatgata gagtcaagtg aataagtgca tcaggcggat gccttggcga tgataggcga
    61 cgaaggacgt gtaagcctgc gaaaagcgcg ggggagctgg caataaagca atgatcccgc
  121 ggtgtccgaa tggggaaacc cactgcattc tgtgcagtat cctaagttga atacataggc
  181 ttagagaagc gaacccggag aactgaacca tctaagtacc cggaggaaaa gaaatcaacc
  241 gagattccgc aagtagtggc gagcgaacgc ggaggagcct gtacgtaata actgtcgagg
  301 tagaagaaca agctgggaag cttgaccata gcgggtgaca gtcccgtatt cgaaatctca
  361 acagcggtac taagcgtacg aaaagtaggg cgggacacgt gaaatcctgt ctgaatatgg
  421 ggggaccatc ctccaaggct aaatactcat catcgaccga tagtgaacca gtaccgtgag
  481 ggaaaggcga aaagaacccc gggaggggag tgaaacagaa cctgaaacct gatgcataca
  541 aacagtggga gcgccctagt ggtgtgactg cgtacctttt gtataatggg tcaacgactt
  601 acattcagta gcgagcttaa ccggataggg gaggcgtagg gaaaccgagt cttaataggg
  661 cgatgagttg ctgggtgtag acccgaaacc gagtgatcta tccatggcca ggttgaaggt
  721 gccgtaacag gtactggagg accgaaccca cgcatgttgc aaaatgcggg gatgagctgt
  781 gggtaggggt gaaaggctaa acaaactcgg agatagctgg ttctccccga aaactattta
  841 ggtagtgcct cgagcaagac actgatgggg gtaaagcact gttatggcta gggggttatt
  901 gcaacttacc aacccatggc aaactcagaa taccatcaag tggttcctcg ggagacagac
  961 agcgggtgct aacgtccgtt gtcaagaggg aaacaaccca gaccgccggc taaggtccca
1021 aatgatagat taagtggtaa acgaagtggg aaggcacaga cagccaggat gttggcttag
1081 aagcagccat catttaaaga aagcgtaata gctcactggt cgagtcgtcc tgcgcggaag
1141 atgtaacggg gctcaaatct ataacccaag ctgcgtatgc cggtttaccg gcatggtagg
1201 ggagcgttct gtaggctgat gaaggtgcat tgtaaagtgt gctggaggta tcagaagtgc
1261 gaatgttgac atgagtagcg ataaagcggg tgaaaagccc gctcgccgca aagcccaagg
1321 tttcctacgc aacgttcatc ggcgtagggt gagtcggccc ctaaggcgag gcagaaatgc
1381 gtagtcgatg ggaaacaggt taatattcct gtacttgatt caaatgcgat gtggggacgg
1441 agaaggttag gttggcaagc tgttggaata gcttgtttaa gccggtaggt ggaagactta
1501 ggcaaatccg ggttttctta acaccgagaa gtgatgacga gtgtctacgg acacgaagca
1561 accgatacca cgcttccagg aaaagccact aagcttcagt ttgaatcgaa ccgtaccgca
1621 aaccgacaca ggtgggcagg atgagaattc taaggcgctt gagagaactc gggagaagga
1681 actcggcaaa ttgataccgt aacttcggga gaaggtatgc cctctaaggt taaggacttg
1741 ctccgtaagc cccggagggt cgcagagaat aggtggctgc gactgtttat taaaaacaca
1801 gcactctgcc aacacgaaag tggacgtata gggtgtgacg cctgcccggt gccggaaggt
1861 taattgaaga tgtgcaagca tcggatcgaa gccccggtaa acggcggccg taactataac
1921 ggtcctaagg tagcgaaatt ccttgtcggg taagttccga cccgcacgaa tggcgtaacg
1981 atggccacac tgtctcctcc cgagactcag cgaagttgaa gtggttgtga agatgcaatc

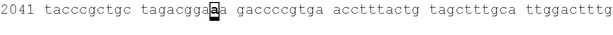

2101 aagtcacttg tgtaggatag gtgggaggct tggaagcaga gacgccagtc tctgtggagt
2161 cgtccttgaa ataccaccct ggtgtctttg aggttctaac ccagacccgt catccgggtc
2221 ggggaccgtg catggtaggc agtttgactg gggcggtctc ctcccaaagc gtaacggagg
2281 agttcgaagg ttacctaggt ccggtcggaa atcggactga tagtgcaatg gcaaaaggta
2341 gcttaactgc gagaccgaca agtcgggcag gtgcgaaagc aggacatagt gatccggtgg
2401 ttctgtatgg aagggccatc gctcaacgga taaaaggtac tccggggata acaggctgat
2461 tccgcccaag agttcatatc gacggcggag tttggcacct cgatgtcggc tcatcacatc
2521 ctggggctgt agtcggtccc aagggtatgg ctgttcgcca tttaaagtgg tacgtgagct

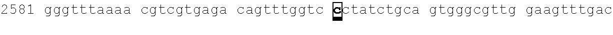

2641 gggggctgct cctagtacga gaggaccgga gtggacgaac ctctggtgta ccggttgtaa
2701 cgccagttgc atagccgggt agctaagttc ggaagagata agcgctgaaa gcatctaagc
2761 gcgaaactcg cctgaagatg agacttccct tgcggtttaa ccgcactaaa gggtcgttcg
2821 agaccaggac gttgataggt ggggtgtgga agcgcggtaa cgcgtgaagc taacccatac
2881 taattgcccg tgaggcttga ctct
23S rRNA allele 3 nucleotide sequence (NCBI GeneID: 3370845; Gene
symbol: NGO r08)
(SEQ ID NO: 23)
      1 tgaaatgata gagtcaagtg aataagtgca tcaggcggat gccttggcga tgataggcga
    61 cgaaggacgt gtaagcctgc gaaaagcgcg ggggagctgg caataaagca atgatcccgc
  121 ggtgtccgaa tggggaaacc cactgcattc tgtgcagtat cctaagttga atacataggc
  181 ttagagaagc gaacccggag aactgaacca tctaagtacc cggaggaaaa gaaatcaacc
  241 gagattccgc aagtagtggc gagcgaacgc ggaggagcct gtacgtaata actgtcgagg
  301 tagaagaaca agctgggaag cttgaccata gcgggtgaca gtcccgtatt cgaaatctca
  361 acagcggtac taagcgtacg aaaagtaggg cgggacacgt gaaatcctgt ctgaatatgg
  421 ggggaccatc ctccaaggct aaatactcat catcgaccga tagtgaacca gtaccgtgag
  481 ggaaaggcga aaagaacccc gggaggggag tgaaacagaa cctgaaacct gatgcataca
  541 aacagtggga gcgccctagt ggtgtgactg cgtacctttt gtataatggg tcaacgactt
  601 acattcagta gcgagcttaa ccggataggg gaggcgtagg gaaaccgagt cttaataggg
  661 cgatgagttg ctgggtgtag acccgaaacc gagtgatcta tccatggcca ggttgaaggt
  721 gccgtaacag gtactggagg accgaaccca cgcatgttgc aaaatgcggg gatgagctgt
  781 gggtaggggt gaaaggctaa acaaactcgg agatagctgg ttctccccga aaactattta
  841 ggtagtgcct cgagcaagac actgatgggg gtaaagcact gttatggcta gggggttatt
  901 gcaacttacc aacccatggc aaactcagaa taccatcaag tggttcctcg ggagacagac
  961 agcgggtgct aacgtccgtt gtcaagaggg aaacaaccca gaccgccggc taaggtccca
1021 aatgatagat taagtggtaa acgaagtggg aaggcacaga cagccaggat gttggcttag
1081 aagcagccat catttaaaga aagcgtaata gctcactggt cgagtcgtcc tgcgcggaag
1141 atgtaacggg gctcaaatct ataaccgaag ctgcggatgc cggtttaccg gcatggtagg
1201 ggagcgttct gtaggctgat gaaggtgcat tgtaaagtgt gctggaggta tcagaagtgc
1261 gaatgttgac atgagtagcg ataaagcggg tgaaaagccc gctcgccgaa agcccaaggt
1321 ttcctacgca acgttcatcg gcgtagggtg agtcggcccc taaggcgagg cagaaatgcg
1381 tagtcgatgg gaaacaggtt aatattcctg tacttgattc aaatgcgatg tggggacgga
1441 gaaggttagg ttggcaagct gttggaatag cttgtttaag ccggtaggtg gaagacttag
1501 gcaaatccgg gttttcttaa caccgagaag tgatgacgag tgtctacgga cacgaagcaa
1561 ccgataccac gcttccagga aaagccacta agcttcagtt tgaatcgaac cgtaccgcaa
1621 accgacacag gtgggcagga tgagaattct aaggcgcttg agagaactcg ggagaaggaa
1681 ctcggcaaat tgataccgta acttcgggag aaggtatgcc ctctaaggtt aaggacttgc
1741 tccgtaagcc ccggagggtc gcagagaata ggtggctgcg actgtttatt aaaaacacag
1801 cactctgcca acacgaaagt ggacgtatag ggtgtgacgc ctgcccggtg ccggaaggtt
1861 aattgaagat gtgcaagcat cggatcgaag ccccggtaaa cggcggccgt aactataacg
1921 gtcctaaggt agcgaaattc cttgtcgggt aagttccgac ccgcacgaat ggcgtaacga
1981 tggccacact gtctcctccc gagactcagc gaagttgaag tggttgtgaa gatgcaatct

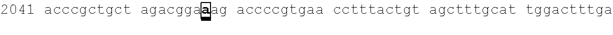

2101 agtcacttgt gtaggatagg tgggaggctt ggaagcagag acgccagtct ctgtggagtc
2161 gtccttgaaa taccaccctg gtgtctttga ggttctaacc cagacccgtc atccgggtcg
2221 gggaccgtgc atggtaggca gtttgactgg ggcggtctcc tcccaaagcg taacggagga
2281 gttcgaaggt tacctaggtc cggtcggaaa tcggactgat agtgcaatgg caaaaggtag
2341 cttaactgcg agaccgacaa gtcgggcagg tgcgaaagca ggacatagtg atccggtggt
2401 tctgtatgga agggccatcg ctcaacggat aaaaggtact ccggggataa caggctgatt
2461 ccgcccaaga gttcatatcg acggcggagt ttggcacctc gatgtcggct catcacatcc
2521 tggggctgta gtcggtccca agggtatggc tgttcgccat ttaaagtggt acgtgagctg

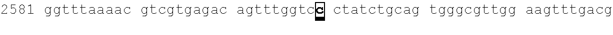

2641 ggggctgctc ctagtacgag aggaccggag tggacgaacc tctggtgtac cggttgtaac
2701 gccagttgca tagccgggta gctaagttcg gaagagataa gcgctgaaag catctaagcg
2761 cgaaactcgc ctgaagatga gacttccctt gcggtttaac cgcactaaag ggtcgttcga
2821 gaccaggacg ttgataggtg gggtgtggaa gcgcggtaac gcgtgaagct aacccatact
2881 aattgcccgt gaggcttgac tct
23S rRNA allele 4 nucleotide sequence (NCBI GeneID: 3370846; Gene
symbol: NGO r11)
(SEQ ID NO: 24)
      1 tgaaatgata gagtcaagtg aataagtgca tcaggcggat gccttggcga tgataggcga
    61 cgaaggacgt gtaagcctgc gaaaagcgcg ggggagctgg caataaagca atgatcccgc
  121 ggtgtccgaa tggggaaacc cactgcattc tgtgcagtat cctaagttga atacataggc
  181 ttagagaagc gaacccggag aactgaccca tctaagtacc cggaggaaaa gaaatcaacc
  241 gagattccgc aagtagtggc gagcgaacgc ggaggagcct gtacgtaata actgtcgagg
  301 tagaagaaca agctgggaag cttgaccata gcgggtgaca gtcccgtatt cgaaatctca
  361 acagcggtac taagcgtacg aaaagtaggg cgggacacgt gaaatcctgt ctgaatatgg
  421 ggggaccatc ctccaaggct aaatactcat catcgaccga tagtgaacca gtaccgtgag
  481 ggaaaggcga aaagaacccc gggaggggag tgaaacagaa cctgaaacct gatgcataca
  541 aacagtggga gcgccctagt ggtgtgactg cgtacctttt gtataatggg tcaacgactt
  601 acattcagta gcgagcttaa ccggataggg gaggcgtagg gaaaccgagt cttaataggg
  661 cgatgagttg ctgggtgtag acccgaaacc gagtgatcta tccatggcca ggttgaaggt
  721 gccgtaacag gtactggagg accgaaccca cgcatgttgc aaaatgcggg gatgagctgt
  781 gggtaggggt gaaaggctaa acaaactcgg agatagctgg ttctccccga aaactattta
  841 ggtagtgcct cgagcaagac actgatgggg gtaaagcact gttatggcta gggggttatt
  901 gcaacttacc aacccatggc aaactcagaa taccatcaag tggttcctcg ggagacagac
  961 agcgggtgct aacgtccgtt gtcaagaggg aaacaaccca gaccgccggc taaggtccca
1021 aatgatagat taagtggtaa acgaagtggg aaggcacaga cagccaggat gttggcttag
1081 aagcagccat catttaaaga aagcgtaata gctcactggt cgagtcgtcc tgcgcggaag
1141 atgtaacggg gctcaaatct ataaccgaag ctgcggatgc cggtttaccg gcatggtagg
1201 ggagcgttct gtaggctgat gaaggtgcat tgtaaagtgt gctggaggta tcagaagtgc
1261 gaatgttgac atgagtagcg ataaagcggg tgaaaagccc gctcgccgaa agcccaaggt
1321 ttcctacgca acgttcatcg gcgtagggtg agtcggcccc taaggcgagg cagaaatgcg
1381 tagtcgatgg gaaacaggtt aatattcctg tacttgattc aaatgcgatg tggggacgga
1441 gaaggttagg ttggcaagct gttggaatag cttgtttaag ccggtaggtg gaagacttag
1501 gcaaatccgg gttttcttaa caccgagaag tgatgacgag tgtctacgga cacgaagcaa
1561 ccgataccac gcttccagga aaagccacta agcttcagtt tgaatcgaac cgtaccgcaa
1621 accgacacag gtgggcagga tgagaattct aaggcgcttg agagaactcg ggagaaggaa
1681 ctcggcaaat tgataccgta acttcgggag aaggtatgcc ctctaaggtt aaggacttgc
1741 tccgtaagcc ccggagggtc gcagagaata ggtggctgcg actgtttatt aaaaacacag
1801 cactctgcca acacgaaagt ggacgtatag ggtgtgacgc ctgcccggtg ccggaaggtt
1861 aattgaagat gtgcaagcat cggatcgaag ccccggtaaa cggcggccgt aactataacg
1921 gtcctaaggt agcgaaattc cttgtcgggt aagttccgac ccgcacgaat ggcgtaacga
1981 tggccacact gtctcctccc gagactcagc gaagttgaag tggttgtgaa gatgcaatct

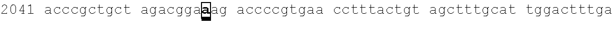

2101 agtcacttgt gtaggatagg tgggaggctt ggaagcagag acgccagtct ctgtggagtc
2161 gtccttgaaa taccaccctg gtgtctttga ggttctaacc cagacccgtc atccgggtcg
2221 gggaccgtgc atggtaggca gtttgactgg ggcggtctcc tcccaaagcg taacggagga
2281 gttcgaaggt tacctaggtc cggtcggaaa tcggactgat agtgcaatgg caaaaggtag
2341 cttaactgcg agaccgacaa gtcgggcagg tgcgaaagca ggacatagtg atccggtggt
2401 tctgtatgga agggccatcg ctcaacggat aaaaggtact ccggggataa caggctgatt
2461 ccgcccaaga gttcatatcg acggcggagt ttggcacctc gatgtcggct catcacatcc
2521 tggggctgta gtcggtccca agggtatggc tgttcgccat ttaaagtggt acgtgagctg

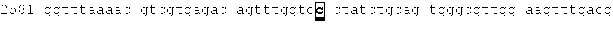

2641 ggggctgctc ctagtacgag aggaccggag tggacgaacc tctggtgtac cggttgtaac
2701 gccagttgca tagccgggta gctaagttcg gaagagataa gcgctgaaag catctaagcg
2761 cgaaactcgc ctgaagatga gacttccctt gcggtttaac cgcactaaag ggtcgttcga
2821 gaccaggacg ttgataggtg gggtgtggaa gcgcggtaac gcgtgaagct aacccatact
2881 aattgcccgt gaggcttgac tct
mtrR gene: nucleotide sequence (NCBI GeneID: 3281546; Gene symbol:
NGO1366)
(SEQ ID NO: 25)
      1 atgagaaaaa ccaaaaccga agccttgaaa accaaagaac acctgatgct tgccgccttg
    61 gaaacctttt accgcaaagg gattgcccgc acctcgctca acgaaatcgc ccaagccgcc

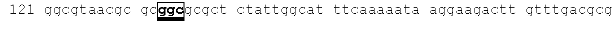

  181 ttgttccaac gtatctgcga cgacatcgaa aactgcatcg cgcaagatgc cgcagatgcc
  241 gaaggaggtt cttggacggt attccgccac acgctgctgc actttttcga gcggctgcaa
  301 agcaacgaca tccactacaa attccacaac atcctgtttt taaagtgcga acatacggaa
  361 caaaacgccg ccgttatcgc cattgcccgc aagcatcagg caatctggcg cgagaaaatt
  421 accgccgttt tgaccgaagc ggtggaaaat caggatttgg ctgacgattt ggacaaggaa
  481 acggcggtca tcttcatcaa atcgacgttg gacgggctga tttggcgttg gttctcttcc
  541 ggcgaaagtt tcgatttggg caaaaccgcc ccgcgcatca tcgggataat gatggacaac
  601 ttggaaaacc atccctgcct gcgccggaaa taa
mtrR gene: protein sequence (GenBank accession no. AAW90014)
(SEQ ID NO: 26)

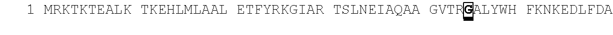

    61 LFQRICDDIE NCIAQDAADA EGGSWTVFRH TLLHFFERLQ SNDIHYKFHN ILFLKCEHTE
  121 QNAAVIAIAR KHQAIWREKI TAVLTEAVEN QDLADDLDKE TAVIFIKSTL DGLIWRWFSS
  181 GESFDLGKTA PRIIGIMMDN LENHPCLRRK
mtrR promoter region of Neisseria qonorrhoeae strain FA19

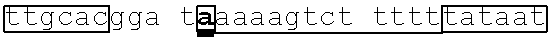

```

In the mtrR promoter region sequence above (SEQ ID NO:27), the −10 and −35 hexamers are shown inside the boxes, the 13-base pair inverted repeat is shown underlined, and the position of the single ‘A’ nucleotide deletion is shown highlighted (Zarantonelli et al., Antimicrobial Agents and Chemotherapy, 1999, 43(10):2468-2472).

Embodiments of the invention are described below, with reference to the accompanying drawings in which:

FIG. 1A shows a sequence alignment of nucleotides 1590 to 1660 of over 100 penA sequences. Residues differing from the wild-type are highlighted. The locations of conserved mutations in penA mutants are shown. FIG. 1B shows the primary nucleotide sequence of the region to target for detection of penA wild-type sequence. Residues that are mutated in mosaic penA alleles are highlighted;

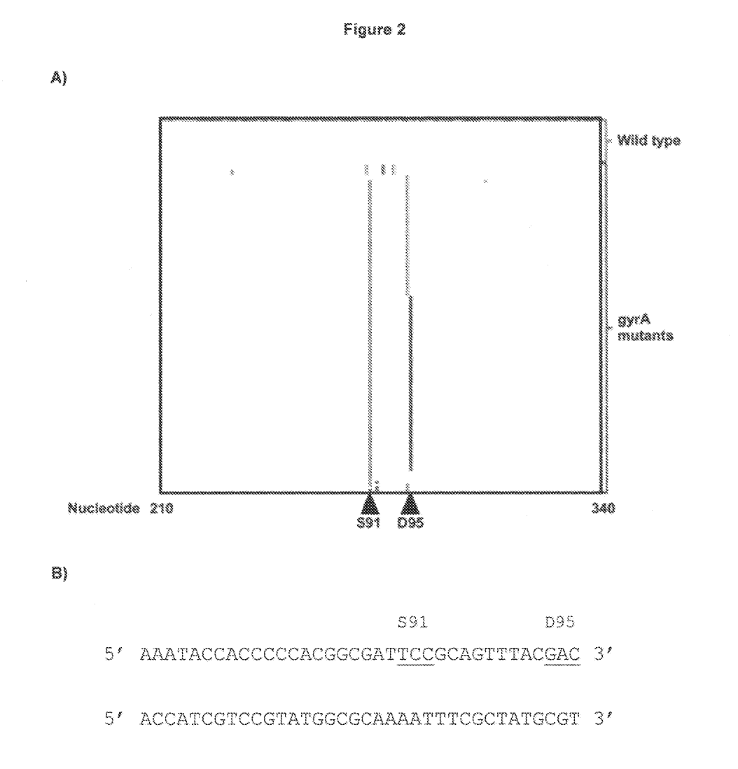

FIG. 2A shows a sequence alignment from nucleotide 210 to 340 of approximately 150 gyrA sequences of Gonorrhoea. Residues differing from the wild-type are highlighted. The locations of conserved mutations in gyrA mutants are shown. FIG. 2B shows the primary nucleotide sequence of the region to target for detection of gyrA wild-type sequence. Residues that are mutated in Ciprofloxacin-resistant gyrA mutants are highlighted; and

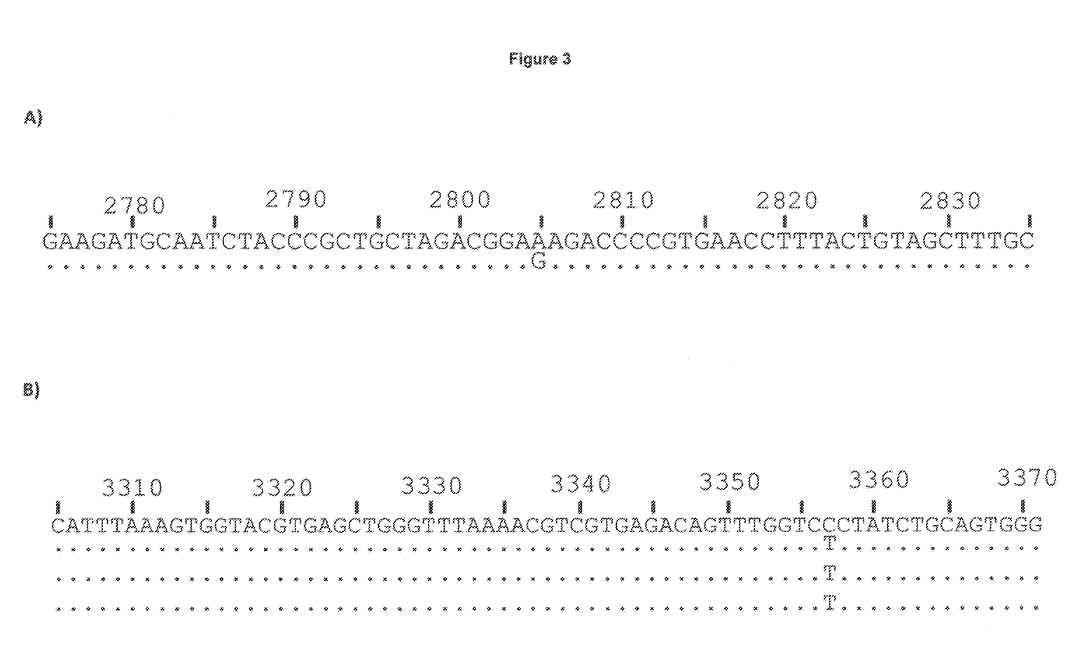

FIG. 3A shows a sequence alignment of wild-type with A2059G mutant, the mutation is shown in the second line (all other nucleotides are the same between the wild type and mutant in this region). FIG. 3B shows a sequence alignment of wild type with C2611T mutants, the mutation is shown in the 2nd, 3rd and 4th lines. All other nucleotides are the same between the wild type and mutants in this region.

### EXAMPLE 1

Cephalosporin Susceptibility Testing

Several mutations in the penA gene have been implicated in extended spectrum Cephalosporin resistance in Gonorrhoea, of which the penA mosaic allele is thought to be of significant relevance. Mosaic penA comprises several regions from a number of different Neisseria species, likely acquired by Neisseria gonorrhoeae through genetic transformation. Over 30 mosaic alleles are in circulation, each of which varies in the number and identity of mutations relative to the wild type Gonorrhoea sequence. However, certain mutations are conserved amongst the majority of penA mosaic alleles.

Determination of susceptibility to ESCs would allow patients that are not resistant to be treated with Cefixime or Ceftriaxone alone, thus giving additional treatment options or allowing monotherapy for significant numbers of patients. It appears that mosaic penA is the only significant determinant in the development of Cefixime resistance, yet there is no single mosaic allele that definitively confers resistance. However, we have appreciated that by identifying patients with wild-type penA sequences, it is possible to identify all patients that are definitely susceptible to Cefixime treatment.

Ceftriaxone resistance mechanisms are significantly more complex than those for Cefixime, Similarly to Cefixime, the presence of a penA mosaic allele is a major factor in the development of resistance. The presence of any one of the more than 30 penA mosaic alleles does not guarantee resistance; rather resistance is dependent on a complex synergy of mutations in the penA, mtrR and porB genes. However, all Gonorrhoea strains identified to date with high-level Ceftriaxone resistance have a mosaic penA. We have appreciated, therefore, that identification of patients with wild-type penA allows the determination of all patients that could be effectively treated with Ceftriaxone.

There is significant diversity in the penA mosaic allele. Over 30 different sequences have been identified to date. To detect the wild-type penA gene in as many cases as possible, oligonucleotides are used which target wild-type residues for which mutations are conserved between the majority of penA mosaic alleles. This allows specific detection of the wild-type, and prevents cross-reaction against Gonorrhoea mutants.

FIG. 1A shows an alignment of over 100 penA nucleotide sequences, including both wild-type and mosaic alleles. The vertical lines indicate positions that are mutated in the mosaic alleles. The F504L and A510V mutation is present in almost all mosaic alleles, whilst A501 and A516 mutations are present in a smaller subset of Gonorrhoea strains.

FIG. 1B shows wild-type nucleotide sequence of the regions shown in FIG. 1A in which mutations are present in the majority of penA mosaic alleles. The locations of mutations are underlined. This is the only region in which mutations are present in the majority of penA mosaic alleles, so this is the region to target for the specific detection of wild-type sequences. Detecting other regions would not allow differentiation between wild-type and certain mutant alleles.

### EXAMPLE 2

Ciprofloxacin Susceptibility/Resistance Testing

Quinolones such as Ciprofloxacin act by inhibiting the activity of two enzymes, DNA gyrase and topoisomerase IV, required for DNA metabolism. Resistance to quinolones developed through the acquisition of single nucleotide polymorphisms (SNPs) in the genes encoding DNA gyrase and topoisomerase IV (gyrA and parC, respectively). Specific SNPs (at S91 and D95) in gyrA alone are sufficient to elicit low- to intermediate-level resistance. High-level resistance requires mutations in both gyrA and parC.

Identification of patients with wild-type gyrA would enable the identification of patients with Gonorrhoea infections that are susceptible to treatment with Ciprofloxacin. This is likely to account for around 50% of patients and will enable the use of cheaper antibiotics, whilst preserving use of drugs such as the ESCs as treatment options for as long as possible.

FIG. 2A shows an alignment of approximately 150 gyrA sequences, including wild-type and mutant sequences. Vertical lines show the nucleotides that are mutated in the gyrA mutants. These are the only mutations in gyrA that are linked with resistance to Ciprofloxacin, so this is the region to target for the specific detection of wild-type gyrA.

FIG. 2B shows wild-type nucleotide sequence of the region shown in FIG. 2A that is mutated in resistant Gonorrhoea strains. The locations of mutations are underlined.

Targeting this region will enable the specific detection of wild-type Gonorrhoea, whilst preventing cross-reaction against mutant strains.

### EXAMPLE 3

Macrolide Resistance Testing (Azithromycin)

Knowing the resistance of Gonorrhoea to Azithromycin is of interest for three reasons: 1) Azithromycin is the recommended treatment for Chlamydia infection, which is frequently found in Gonorrhoea positive patients, 2) Azithromycin is administered in conjunction with Ceftriaxone in many developed countries to ensure treatment is successful; 3) knowing whether patients are infected with Azithromycin susceptible Gonorrhoea could allow it to be used alone for a percentage of patients, thus preserving ESCs as a treatment option. Azithromycin acts by binding to the 23S ribosomal RNA (rRNA), part of the 50S subunit, which leads to inhibition of bacterial protein synthesis. Resistance to Azithromycin can occur by three mechanisms: 1) Methylase modification of 23S rRNA; 2) Overexpression of efflux pumps, which can act to increase the removal of antibiotics from the cell; 3) SNP of particular nucleotides of the 23S rRNA.

Nucleic acid testing is only able to detect resistance that arises as a result of SNPs in the 23S rRNA sequence. However, methylase modifications are very rare in Azithromycin strains.

Specific point mutations of the Azithromycin target, the 23S rRNA, can result in varying degrees of resistance (C2611T—low level resistance; A2059G—high-level resistance). The level of Azithromycin resistance is also linked to the number of mutated 23S alleles—Neisseria gonorrhoeae has four copies of the 23S rRNA gene. If mutation is observed in only one of four of the alleles, even if the mutation is A2059G, low levels of resistance will be observed. However, strains with a single mutated allele, while susceptible to treatment will quickly develop high-level resistance.

Targeting these point mutations to determine strains that could be ‘high-risk’ for treatment with Azithromycin allows different antibiotics to be selected for treatment.

1. A method for determining whether a subject suffering from, or suspected of suffering from, an infectious disease caused by a microbe is infected with a strain of the microbe that is susceptible to an antimicrobial agent, wherein there exist different strains of the microbe that are resistant to the antimicrobial agent, wherein the method comprises determining whether nucleic acid of the strain of the microbe infecting the subject comprises wild-type nucleotide sequence at a conserved nucleotide position at which mutation is associated with resistance to the antimicrobial agent in nucleic acid of the different resistant strains.

2. A method according to claim 1, wherein it is determined whether nucleic acid of the strain infecting the subject comprises wild-type nucleotide sequence at a first conserved nucleotide position, and at a second, different conserved nucleotide position, wherein the first conserved nucleotide position is mutated in a first subset of strains of the microbe that are resistant to the antimicrobial agent, and the second conserved nucleotide position is mutated in a second subset of strains of the microbe that are resistant to the antimicrobial agent.

3. A method according to claim 1 or 2, wherein the antimicrobial agent is a first antimicrobial agent, and the method further comprises determining whether the subject is infected with a strain of the microbe that is susceptible to a second antimicrobial agent, wherein there exist different strains of the microbe that are resistant to the second antimicrobial agent, and wherein the method comprises determining whether nucleic acid of the strain of the microbe infecting the subject comprises wild-type nucleotide sequence at a conserved nucleotide position at which mutation is associated with resistance to the second antimicrobial agent in nucleic acid of the different resistant strains.

4. A method according to claim 3, wherein it is determined whether the subject is infected with a strain of the microbe that is susceptible to the second antimicrobial agent if it is determined that the subject is infected with a strain of the microbe that is resistant to the first antimicrobial agent.

5. A method according to any preceding claim, which comprises determining whether the strain of the microbe infecting the subject comprises the wild-type nucleotide sequence by specifically detecting for the wild-type nucleotide sequence.

6. A method according to claim 5, which comprises specifically detecting for the wild-type nucleotide sequence using a method that comprises amplification of nucleic acid of the strain infecting the subject that has been obtained from the subject.

7. A method according to claim 6, which further comprises detecting for product resulting from amplification of the nucleic acid using a dipstick.

8. A method according to any of claims 5 to 7, which comprises specifically detecting for the wild-type nucleotide sequence using an oligonucleotide that hybridizes under stringent conditions to nucleic acid comprising sequence that is the same sequence as, or complementary to, the wild-type nucleotide sequence, but which does not hybridize under stringent conditions to nucleic acid comprising sequence that is the same sequence as, or complementary to, nucleotide sequence comprising a mutation at the conserved nucleotide position that is associated with resistance to the antimicrobial agent.

9. A method according to any preceding claim, wherein the infectious disease is a sexually transmitted disease.

10. A method according to any preceding claim, wherein the infectious disease is Gonorrhoea.

11. A method according to claim 10, for determining whether a subject suffering from Gonorrhoea is infected with an antibiotic-susceptible strain of Neisseria gonorrhoeae, which comprises determining whether the strain of Neisseria gonorrhoeae comprises wild-type nucleotide sequence at a conserved nucleotide position of the penA mosaic gene, the gyrA gene, or 23S ribosomal RNA.

12. A method according to claim 11 for determining whether the subject is infected with a Cephalosporin-susceptible strain of Neisseria gonorrhoeae, wherein it is determined whether the strain of Neisseria gonorrhoeae comprises wild-type nucleotide sequence encoding position F504 and/or A510 of the penA mosaic gene.

13. A method according to claim 12, which further comprises determining whether the strain of Neisseria gonorrhoeae comprises wild-type nucleotide sequence encoding position A501 and/or A516 of the penA mosaic gene.

14. A method according to claim 10, for determining whether a subject suffering from Gonorrhoea is infected with a Cephalosporin-susceptible strain of Neisseria gonorrhoeae, which comprises determining whether the strain of Neisseria gonorrhoeae comprises wild-type nucleotide sequence encoding a conserved position of the penA non-mosaic gene, preferably position A501 of the penA non-mosaic gene.

15. A method according to any of claims 11 to 14, for determining whether the subject is infected with a Ciprofloxacin-susceptible strain of Neisseria gonorrhoeae, wherein it is determined whether the strain of Neisseria gonorrhoeae comprises wild-type nucleotide sequence encoding position S91 and/or D95 of the gyrA gene.

16. A method according to any of claims 11 to 15, for determining whether the subject is infected with an Azithromycin-susceptible strain of Neisseria gonorrhoeae, wherein it is determined whether the strain of Neisseria gonorrhoeae comprises wild-type nucleotide sequence encoding position C2611 and/or A2059 of 23S ribosomal RNA, and optionally whether the strain of Neisseria gonorrhoeae does not comprise mutant nucleotide sequence encoding position C2611 and/or A2059 of 23S ribosomal RNA.

17. A method according to claim 16, which further comprises determining whether the strain of N. gonorrhoeae comprises a wild-type mtrR promoter sequence and/or a wild-type nucleotide sequence encoding position G45 of the mtrR gene.

18. A method according to claim 11, which comprises:
i) determining whether the subject is infected with a strain of Neisseria gonorrhoeae that is susceptible to a first antimicrobial agent selected from Cephalosporin, Ciprofloxacin, and Azithromycin, using a method according to any of claims 11 to 17; and, if it is determined that the subject is infected with a strain of Neisseria gonorrhoeae that is resistant to the first antimicrobial agent,
ii) determining whether the subject is infected with a strain of Neisseria gonorrhoeae that is susceptible to a second, different antimicrobial agent selected from Cephalopsorin, Ciprofloxacin, and Azithromycin, using a method according to any of claims 11 to 17; and, if it is determined that the subject is infected with a strain of Neisseria gonorrhoeae that is resistant to the second antimicrobial agent,
iii) determining whether the subject is infected with a strain of Neisseria gonorrhoeae that is susceptible to a third, different antimicrobial agent selected from Cephalopsorin, Ciprofloxacin, and Azithromycin, using a method according to any of claims 11 to 17.

19. A method of treating, or prescribing treatment of, a subject suffering from, or suspected of suffering from, an infectious disease caused by a microbe, which comprises:
determining whether the subject is infected with a strain of the microbe that is susceptible to an antimicrobial agent, wherein there exist different strains of the microbe that are resistant to the antimicrobial agent, using a method according to any of claims 1 to 8; and
administering, or prescribing for administration, to the subject an effective amount of the antimicrobial agent as a monotherapy if it is determined that the subject is infected with a strain of the microbe that is susceptible to the antimicrobial agent.

20. A method according to claim 19, which comprises:
determining whether the subject is infected with a strain of the microbe that is susceptible to a first antimicrobial agent, wherein there exist different strains of the microbe that are resistant to the first antimicrobial agent, using a method according to any of claims 1 to 8; and
administering, or prescribing for administration, to the subject an effective amount of the first antimicrobial agent as a monotherapy if it is determined that the subject is infected with a strain of the microbe that is susceptible to the first antimicrobial agent; or
if it is determined that the subject is infected with a strain of the microbe that is resistant to the first antimicrobial agent, determining whether the subject is infected with a strain of the microbe that is susceptible to a second, different antimicrobial agent, wherein there exist different strains of the microbe that are resistant to the second antimicrobial agent, using a method according to any of claims 1 to 8; and
administering, or prescribing for administration, to the subject an effective amount of the second antimicrobial agent as a monotherapy if it is determined that the subject is infected with a strain of the microbe that is susceptible to the second antimicrobial agent; or
if it is determined that the subject is infected with a strain of the microbe that is resistant to the second antimicrobial agent, administering, or prescribing for administration, to the subject an effective amount of the first and the second antimicrobial agent as a combination therapy.

21. A method according to claim 19, wherein the infectious disease is Gonorrhoea, and wherein the method comprises:
determining whether the subject is infected with an antibiotic-susceptible strain of Neisseria gonorrhoeae by determining whether the strain of N. gonorrhoeae comprises wild-type nucleotide sequence encoding the penA mosaic gene, the gyrA gene, or 23S ribosomal RNA; and
administering, or prescribing for administration, Cephalosporin to the subject as a monotherapy if it is determined that the strain of N. gonorrhoeae comprises wild-type nucleotide sequence encoding the penA mosaic gene;
administering, or prescribing for administration, Ciprofloxacin to the subject as a monotherapy if it is determined that the strain of N. gonorrhoeae comprises wild-type nucleotide sequence encoding the gyrA gene; or
administering, or prescribing for administration, Azithromycin to the subject as a monotherapy if it is determined that the strain of N. gonorrhoeae comprises wild-type nucleotide sequence of 23S ribosomal RNA.

22. A method according to claim 19, wherein the infectious disease is Gonorrhoea, and wherein the method comprises:
determining whether the subject is infected with an antibiotic-susceptible strain of Neisseria gonorrhoeae by determining whether the strain of N. gonorrhoeae comprises wild-type nucleotide sequence encoding the penA mosaic gene, the penA non-mosaic gene, the gyrA gene, or 23S ribosomal RNA and, optionally, the mtrR gene and/or the mtrR promoter; and
administering, or prescribing for administration, Cephalosporin to the subject as a monotherapy if it is determined that the strain of N. gonorrhoeae comprises wild-type nucleotide sequence encoding the penA mosaic gene, or the penA non-mosaic gene;
administering, or prescribing for administration, Ciprofloxacin to the subject as a monotherapy if it is determined that the strain of N. gonorrhoeae comprises wild-type nucleotide sequence encoding the gyrA gene; or
administering, or prescribing for administration, Azithromycin to the subject as a monotherapy if it is determined that the strain of N. gonorrhoeae comprises wild-type nucleotide sequence of 23S ribosomal RNA and, optionally, the mtrR gene and/or the mtrR promoter.

23. A method according to claim 20, wherein the infectious disease is Gonorrhoea, and the first and the second antimicrobial agent are each selected from Cephalosporin, Ciprofloxacin, and Azithromycin, and wherein it is determined whether the subject is infected with a Cephalosporin-susceptible strain by determining whether the strain of N. gonorrhoeae comprises wild-type nucleotide sequence encoding the penA mosaic gene, a Ciprofloxacin-resistant strain by determining whether the strain of N. gonorrhoeae comprises wild-type nucleotide sequence encoding the gyrA gene, and an Azithromycin-resistant strain by determining whether the strain of N. gonorrhoeae comprises wild-type nucleotide sequence of 23S ribosomal RNA.

24. A method according to claim 20, wherein the infectious disease is Gonorrhoea, and the first and the second antimicrobial agent are each selected from Cephalosporin, Ciprofloxacin, and Azithromycin, and wherein it is determined whether the subject is infected with a Cephalosporin-susceptible strain by determining whether the strain of N. gonorrhoeae comprises wild-type nucleotide sequence encoding the penA mosaic gene or the penA non-mosaic gene, a Ciprofloxacin-resistant strain by determining whether the strain of N. gonorrhoeae comprises wild-type nucleotide sequence encoding the gyrA gene, and an Azithromycin-resistant strain by determining whether the strain of N. gonorrhoeae comprises wild-type nucleotide sequence of 23S ribosomal RNA and, optionally, the mtrR gene and/or the mtrR promoter.

25. A method according to any of claims 21 to 24, which comprises determining whether the subject is infected with a Cephalosporin-susceptible strain of Neisseria gonorrhoeae by determining whether the strain of Neisseria gonorrhoeae comprises wild-type nucleotide sequence encoding position F504 and/or A510 of the penA mosaic gene, and administering, or prescribing for administration, an effective amount of Cephalosporin to the subject as a monotherapy if it is determined that the strain of N. gonorrhoeae comprises wild-type nucleotide sequence encoding position F504 and/or A510 of the penA mosaic gene.

26. A method according to claim 25, which further comprises determining whether the strain of Neisseria gonorrhoeae comprises wild-type nucleotide sequence encoding position A501 and/or A516 of the penA mosaic gene, and administering, or prescribing for administration, an effective amount of Cephalosporin to the subject as a monotherapy if it is determined that the strain of N. gonorrhoeae comprises wild-type nucleotide sequence encoding position F504 and/or A510 of the penA mosaic gene.

27. A method according to claim 22 or 24, which comprises determining whether the subject is infected with a Cephalosporin-susceptible strain of Neisseria gonorrhoeae by determining whether the strain of Neisseria gonorrhoeae comprises wild-type nucleotide sequence encoding position A501 of the penA non-mosaic gene, and administering, or prescribing for administration, an effective amount of Cephalosporin to the subject as a monotherapy if it is determined that the strain of N. gonorrhoeae comprises wild-type nucleotide sequence encoding position A501 of the penA non-mosaic gene.

28. A method according to any of claims 21 to 27, which comprises determining whether the subject is infected with a Ciprofloxacin-susceptible strain of Neisseria gonorrhoeae by determining whether the strain of Neisseria gonorrhoeae comprises wild-type nucleotide sequence encoding position S91 and/or D95 of the gyrA gene, and administering, or prescribing for administration, an effective amount of Ciprofloxacin to the subject as a monotherapy if it is determined that the strain of N. gonorrhoeae comprises wild-type nucleotide sequence encoding position S91 and/or D95 of the gyrA gene.

29. A method according to any of claims 21 to 28, which comprises determining whether the subject is infected with an Azithromycin-susceptible strain of Neisseria gonorrhoeae by determining whether the strain of Neisseria gonorrhoeae comprises wild-type nucleotide sequence encoding position C2611 and/or A2059 of 23S ribosomal RNA, and administering, or prescribing for administration, an effective amount of Azithromycin as a monotherapy if it is determined that the strain of N. gonorrhoeae comprises wild-type nucleotide sequence of 23S ribosomal RNA.

30. A method according to claim 29, which further comprises determining whether the strain of Neisseria gonorrhoeae does not comprise mutant nucleotide sequence encoding position C2611 and/or A2059 of 23S ribosomal RNA, and administering, or prescribing for administration, an effective amount of Azithromycin as a monotherapy if it is determined that the strain of N. gonorrhoeae comprises wild-type nucleotide sequence of 23S ribosomal RNA and does not comprise mutant nucleotide sequence of 23S ribosomal RNA.

31. A method according to claim 29 or 30, which further comprises determining whether the strain of N. gonorrhoeae comprises a wild-type mtrR promoter sequence and/or a wild-type nucleotide sequence encoding position G45 of the mtrR gene, and administering, or prescribing for administration, an effective amount of Azithromycin as a monotherapy if it is determined that the strain of N. gonorrhoeae comprises wild-type nucleotide sequence of 23S ribosomal RNA, and optionally does not comprise mutant nucleotide sequence of 23S ribosomal RNA, and that the strain of N. gonorrhoeae comprises a wild-type mtrR promoter sequence and/or a wild-type nucleotide sequence encoding position G45 of the mtrR gene.

32. A kit for determining whether a subject suffering from Gonorrhoea is infected with an antibiotic-susceptible strain of Neisseria gonorrhoeae, which comprises:
i) an oligonucleotide that hybridizes under stringent conditions to N. gonorrhoeae nucleic acid comprising sequence that is the same sequence as, or complementary to, wild-type nucleotide sequence of the penA mosaic gene, wherein the oligonucleotide comprises nucleotide sequence that is complementary to, or the same sequence as, wild-type nucleotide sequence encoding position F504 and/or A510 of the penA mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position F504 and/or A510 of the penA mosaic gene; and/or
ii) an oligonucleotide that hybridizes under stringent conditions to N. gonorrhoeae nucleic acid comprising sequence that is the same sequence as, or complementary to, wild-type nucleotide sequence of the penA mosaic gene, wherein the oligonucleotide comprises nucleotide sequence that is complementary to, or the same sequence as, wild-type nucleotide sequence encoding position A501 and/or A516 of the penA mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position A501 and/or A516 of the penA mosaic gene; and/or
iii) an oligonucleotide that hybridizes under stringent conditions to N. gonorrhoeae nucleic acid comprising sequence that is the same sequence as, or complementary to, wild-type nucleotide sequence of the gyrA gene, wherein the oligonucleotide comprises nucleotide sequence that is complementary to, or the same sequence as, wild-type nucleotide sequence encoding position S91 and/or D95 of the gyrA gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position S91 and/or D95 of the gyrA gene; and/or
iv) an oligonucleotide that hybridizes under stringent conditions to N. gonorrhoeae nucleic acid comprising sequence that is the same sequence as, or complementary to, wild-type nucleotide sequence of 23S ribosomal RNA, wherein the oligonucleotide comprises nucleotide sequence that is complementary to, or the same sequence as, wild-type nucleotide sequence encoding position C2611 and/or A2059 of 23S ribosomal RNA, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position C2611 and/or A2059 of 23S ribosomal RNA.

33. A kit according to claim 32, which comprises the oligonucleotide of (i) and/or (ii) and/or (iii) and/or (iv), and/or:
v) an oligonucleotide that hybridizes under stringent conditions to N. gonorrhoeae nucleic acid comprising sequence that is the same sequence as, or complementary to, wild-type nucleotide sequence of the penA non-mosaic gene, wherein the oligonucleotide comprises nucleotide sequence that is complementary to, or the same sequence as, wild-type nucleotide sequence encoding position A501 of the penA non-mosaic gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position A501 of the penA non-mosaic gene.

34. A kit according to claim 32 or 33 which comprises the oligonucleotide of (iv), and wherein the kit further comprises:
vi) an oligonucleotide that hybridizes under stringent conditions to N. gonorrhoeae nucleic acid comprising sequence that is the same sequence as, or complementary to, wild-type nucleotide sequence from position −10 to −35 of the mtrR promoter, wherein the oligonucleotide comprises nucleotide sequence that is complementary to, or the same sequence as, wild-type nucleotide sequence from −10 to −35 of the mtrR promoter, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae comprising, a mutation at a position from −10 to −35 of the mtrR promoter; and/or
vii) an oligonucleotide that hybridizes under stringent conditions to N. gonorrhoeae nucleic acid comprising sequence that is the same sequence as, or complementary to, wild-type nucleotide sequence encoding position G45 of the mtrR gene, wherein the oligonucleotide comprises nucleotide sequence that is complementary to, or the same sequence as, wild-type nucleotide sequence encoding position G45 of the mtrR gene, and wherein the oligonucleotide does not hybridize under stringent conditions to N. gonorrhoeae nucleic acid comprising sequence that is the same sequence as, or complementary to, nucleotide sequence of a resistant strain of N. gonorrhoeae encoding a mutation at position G45 of the mtrR gene.

35. A kit according to any of claims 32 to 34, wherein the oligonucleotides are selected from oligonucleotides that hybridize under stringent conditions to nucleic acid comprising sequence that is the same sequence as, or complementary to, the nucleotide sequence of:
(SEQ ID NO: 1)
AAACCGGCACGGCGCGCAAGTTCGTCAACGGGCGT;
(SEQ ID NO: 2)
TATGCCGACAACAAACACGTCGCTACCTTTATCGG;
(SEQ ID NO: 3)
AAATACCACCCCCACGGCGATTCCGCAGTTTACGAC;
(SEQ ID NO: 4)
ACCATCGTCCGTATGGCGCAAAATTTCGCTATGCGT;
(SEQ ID NO: 5)
GAAGATGCAATCTACCCGCTGCTAGACGGAAAGACCCCGTGAACCTTTAC
TGTAGCTTTGC;
or
(SEQ ID NO: 6)
CATTTAAAGTGGTACGTGAGCTGGGTTTAAAACGTCGTGAGACAGTTTGG
TCCCTATCTGCAGTGGG.

36. A kit according to any of claims 32 to 34, wherein the kit comprises oligonucleotides selected from oligonucleotides that hybridize under stringent conditions to nucleic acid comprising sequence that is the same sequence as, or complementary to, the nucleotide sequence of:
(SEQ ID NO: 1)
AAACCGGCACGGCGCGCAAGTTCGTCAACGGGCGT;
(SEQ ID NO: 2)
TATGCCGACAACAAACACGTCGCTACCTTTATCGG;
(SEQ ID NO: 3)
AAATACCACCCCCACGGCGATTCCGCAGTTTACGAC;
(SEQ ID NO: 4)
ACCATCGTCCGTATGGCGCAAAATTTCGCTATGCGT;
(SEQ ID NO: 5)
GAAGATGCAATCTACCCGCTGCTAGACGGAAAGACCCCGTGAACCTTTAC
TGTAGCTTTGC;
or
(SEQ ID NO: 6)
CATTTAAAGTGGTACGTGAGCTGGGTTTAAAACGTCGTGAGACAGTTTGG
TCCCTATCTGCAGTGGG;
or
(SEQ ID NO: 7)
AAACCGGCACGGCGCGCAAGTTCGTCAACGGGCGTTATGCCGACAACAAA
CACGTCGCTACCTTTATCGG;
or
(SEQ ID NO: 8)
AAATACCACCCCCACGGCGATTCCGCAGTTTACGACACCATCGTCCGTAT
GGCGCAAAATTTCGCTATGCGT.

37. A kit according to any of claims 32 to 34, wherein the kit comprises oligonucleotides selected from oligonucleotides that hybridize under stringent conditions to nucleic acid comprising sequence that is the same sequence as, or complementary to, the nucleotide sequence of:
(SEQ ID NO: 5)
GAAGATGCAATCTACCCGCTGCTAGACGGAAAGACCCCGTGAACCTTTAC
TGTAGCTTTGC;
(SEQ ID NO: 6)
CATTTAAAGTGGTACGTGAGCTGGGTTTAAAACGTCGTGAGACAGTTTGG
TCCCTATCTGCAGTGGG;
(SEQ ID NO: 7)
AAACCGGCACGGCGCGCAAGTTCGTCAACGGGCGTTATGCCGACAACAAA
CACGTCGCTACCTTTATCGG;
or
(SEQ ID NO: 8)
AAATACCACCCCCACGGCGATTCCGCAGTTTACGACACCATCGTCCGTAT
GGCGCAAAATTTCGCTATGCGT.

38. A kit according to any of claims 32 to 37, which further comprises oligonucleotide primers for amplification of Neisseria gonorrhoeae nucleic acid that comprises the wild-type nucleotide sequence encoding position: A501 and/or A516 of the penA mosaic gene; S91 and/or D95 of the gyrA gene; or C2611 and/or A2059 of 23S ribosomal RNA.

39. A kit according to any of claims 32 to 37, which further comprises oligonucleotide primers for amplification of Neisseria gonorrhoeae nucleic acid that comprises the wild-type nucleotide sequence encoding position: F504 and/or A510 of the penA mosaic gene and optionally, A501 and/or A516 of the penA mosaic gene; S91 and/or D95 of the gyrA gene; or C2611 and/or A2059 of 23S ribosomal RNA.

40. A kit according to any of claims 32 to 38, which further comprises oligonucleotide primers for amplification of Neisseria gonorrhoeae nucleic acid that comprises the wild-type nucleotide sequence encoding position: F504 and/or A510 of the penA mosaic gene and optionally, A501 and/or A516 of the penA mosaic gene; A501 of the penA non-mosaic gene; S91 and/or D95 of the gyrA gene; C2611 and/or A2059 of 23S ribosomal RNA and, optionally, −10 to −35 of the mtrR promoter and/or G45 of the mtrR gene.


SEQUENCE LISTING (27 sequences)

SEQ ID NO: 1 - DNA, 35 bp, Neisseria gonorrhoeae
aaaccggcacggcgcgcaagttcgtcaacgggcgt

SEQ ID NO: 2 - DNA, 35 bp, Neisseria gonorrhoeae
tatgccgacaacaaacacgtcgctacctttatcgg

SEQ ID NO: 3 - DNA, 36 bp, Neisseria gonorrhoeae
aaataccacccccacggcgattccgcagtttacgac

SEQ ID NO: 4 - DNA, 36 bp, Neisseria gonorrhoeae
accatcgtccgtatggcgcaaaatttcgctatgcgt

SEQ ID NO: 5 - DNA, 61 bp, Neisseria gonorrhoeae
gaagatgcaatctacccgctgctagacggaaagaccccgtgaacctttactgtagctttgc

SEQ ID NO: 6 - DNA, 67 bp, Neisseria gonorrhoeae
catttaaagtggtacgtgagctgggtttaaaacgtcgtgagacagtttggtccctatctgcagtggg

SEQ ID NO: 7 - DNA, 70 bp, Neisseria gonorrhoeae
aaaccggcacggcgcgcaagttcgtcaacgggcgttatgccgacaacaaacacgtcgctacctttatcgg

SEQ ID NO: 8 - DNA, 72 bp, Neisseria gonorrhoeae
aaataccacccccacggcgattccgcagtttacgacaccatcgtccgtatggcgcaaaatttcgctatgcgt

SEQ ID NO: 9 - DNA, 24 bp, Artificial
  PCR primer sequence
acgaatggcgtaacgatggccaca

SEQ ID NO: 10 - DNA, 24 bp, Artificial
  PCR primer sequence
tcagaatgccacagcttacaaact

SEQ ID NO: 11 - DNA, 24 bp, Artificial
  PCR primer sequence
gcgaccataccaaacacccacagg

SEQ ID NO: 12 - DNA, 24 bp, Artificial
  PCR primer sequence
gatcccgttgcagtgaagaaagtc

SEQ ID NO: 13 - DNA, 24 bp, Artificial
  PCR primer sequence
aacagacttactatcccattcagc

SEQ ID NO: 14 - DNA, 24 bp, Artificial
  PCR primer sequence
ttcgtccactccggtcctctcgta

SEQ ID NO: 15 - DNA, 29 bp, Artificial
  PCR primer sequence
actgaagcttatttccggcgcaggcaggg

SEQ ID NO: 16 - DNA, 21 bp, Artificial
  PCR primer sequence
gacgacagtgccaatgcaacg

SEQ ID NO: 17 - DNA, 1746 bp, Neisseria gonorrhoeae
atgttgattaaaagcgaatataagccccggatgctgcccaaagaagagcaggtcaaaaagccgatgaccagtaacggacggattagcttcgtcctgatggcaatggcggtcttgtttgcctgtctgattgcccgcgggctgtatctgcagacggtaacgtataactttttgaaagaacagggcgacaaccggattgtgcggactcaagcattgccggctacacgcggtacggtttcggaccggaacggtgcggttttggcgttgagcgcgccgacggagtccctgtttgccgtgcctaaagatatgaaggaaatgccgtctgccgcccaattggaacgcctgtccgagcttgtcgatgtgccggtcgatgttttgaggaacaaactcgaacagaaaggcaagtcgtttatttggatcaagcggcagctcgatcccaaggttgccgaagaggtcaaagccttgggtttggaaaactttgtatttgaaaaagaattaaaacgccattacccgatgggcaacctgtttgcacacgtcatcggatttaccgatattgacggcaaaggtcaggaaggtttggaactttcgcttgaagacagcctgtatggcgaagacggcgcggaagttgttttgcgggaccggcagggcaatattgtggacagcttggactccccgcgcaataaagcaccgcaaaacggcaaagacatcatcctttccctcgatcagaggattcagaccttggcctatgaagagttgaacaaggcggtcgaataccatcaggcaaaagccggaacggtggtggttttggatgcccgcacgggggaaatcctcgccttggccaatacgcccgcctacgatcccaacagacccggccgggcagacagcgaacagcggcgcaaccgtgccgtaaccgatatgatcgaacctggttcggcaatcaaaccgttcgtgattgcgaaggcattggatgcgggcaaaaccgatttgaacgaacggctgaatacgcagccttataaaatcggaccgtctcccgtgcgcgatacccatgtttacccctctttggatgtgcgcggcattatgcagaaatcgtccaacgtcggcacaagcaaactgtctgcgcgtttcggcgccgaagaaatgtatgacttctatcatgaattgggcatcggtgtgcgtatgcactcgggctttccgggggaaactgcaggtttgttgagaaattggcgcaggtggcggcccatcgaacaggcgacgatgtctttcggttacggtctgcaattgagcctgctgcaattggcgcgcgcctataccgcactgacgcacgacggcgttttgctgccgctcagctttgagaagcaggcggttgcgccgcaaggcaaacgcatattcaaagaatcgaccgcgcgcgaggtacgcaatctgatggtttccgtaaccgagccgggcggcaccggtacggcgggtgcggtggacggtttcgatgtcggcgctaaaaccggcacggcgcgcaagttcgtcaacgggcgttatgccgacaacaaacacgtcgctacctttatcggttttgcccccgccaaaaacccccgtgtgattgtggcggtaaccatcgacgaaccgactgcccacggctattacggcggcgtagtggcagggccgcccttcaaaaaaattatgggcggcagcctgaacatcttgggcatttccccgaccaagccactgaccgccgcagccgtcaaaacaccgtcttaa

SEQ ID NO: 18 - protein, 581 aa, Neisseria gonorrhoeae
MLIKSEYKPRMLPKEEQVKKPMTSNGRISFVLMAMAVLFACLIARGLYLQTVTYNFLKEQGDNRIVRTQALPATRGTVSDRNGAVLALSAPTESLFAVPKDMKEMPSAAQLERLSELVDVPVDVLRNKLEQKGKSFIWIKRQLDPKVAEEVKALGLENFVFEKELKRHYPMGNLFAHVIGFTDIDGKGQEGLELSLEDSLYGEDGAEVVLRDRQGNIVDSLDSPRNKAPQNGKDIILSLDQRIQTLAYEELNKAVEYHQAKAGTVVVLDARTGEILALANTPAYDPNRPGRADSEQRRNRAVTDMIEPGSAIKPFVIAKALDAGKTDLNERLNTQPYKIGPSPVRDTHVYPSLDVRGIMQKSSNVGTSKLSARFGAEEMYDFYHELGIGVRMHSGFPGETAGLLRNWRRWRPIEQATMSFGYGLQLSLLQLARAYTALTHDGVLLPLSFEKQAVAPQGKRIFKESTAREVRNLMVSVTEPGGTGTAGAVDGFDVGAKTGTARKFVNGRYADNKHVATFIGFAPAKNPRVIVAVTIDEPTAHGYYGGVVAGPPFKKIMGGSLNILGISPTKPLTAAAVKTPS

SEQ ID NO: 19 - DNA, 2751 bp, Neisseria gonorrhoeae
atgaccgacgcaaccatccgccacgaccacaaattcgccctcgaaaccctgcccgtcagccttgaagacgaaatgcgcaaaagctatctcgactacgccatgagcgtcattgtcgggcgcgcgctgccggacgttcgcgacggcctaaagccggtgcaccggcgcgtactgtacgcgatgcacgagctgaaaaataactggaatgccgcctacaaaaaatcggcgcgcatcgtcggcgacgtcatcggtaaataccacccccacggcgattccgcagtttacgacaccatcgtccgtatggcgcaaaatttcgctatgcgttatgtgctgatagacggacagggcaacttcggatcggtggacgggcttgccgccgcagccatgcgctataccgaaatccgcatggcgaaaatctcacatgaaatgctggcagacattgaggaagaaaccgttaatttcggcccgaactacgacggtagcgaacacgagccgcttgtactgccgacccgtttccccacactgctcgtcaacggctcgtccggtatcgccgtcggtatggcgaccaacatcccgccgcacaacctcaccgacaccatcaacgcctgtctgcgtcttttggacgaacccaaaaccgaaatcgacgaactgatcgacattatccaagcccccgacttcccgaccggggcaaccatctacggcttgggcggcgtgcgcgaaggctataaaacaggccgcggccgcgtcgttatacgcggtaagacccatatcgaacccataggcaaaaacggcgaacgcgaagccatcgttatcgacgaaatcccctatcaggtcaacaaagccaagttggtcgagaaaatcggcgatttggttcgggaaaaaacgctggaaggcatttccgagctccgcgacgaatccgacaaatccgggatgcgcgtcgttatcgagctgaaacgcaacgaaaatgccgaagtcgtcttaaaccaactctacaaactgactccgctgcaagacagtttcggcatcaatatggttgttttggtcgacggacaaccgcgcctgttaaacctgaaacagattctctccgaattcctgcgccaccgccgcgaagtcgttacccgacgtacgcttttccggctgaagaaggcacgccatgaagggcatatcgccgaaggcaaagccgtcgcactgtccaatatcgatgaaatcatcaagctcatcaaagaatcgcccaacgcggccgaggccaaagaaaaactgcttgcgcgcccttggcgcagcagcctcgttgaagaaatgctgacgcgttccggtctggatttggaaatgatgcgtccggaaggattggctgcaaacattggtctgaaaaaacaaggttattacctgagcgagattcaggcagatgctattttacgcatgagcctgcgaaacctgaccggcctcgatcagaaagaaattatcgaaagctacaaaaacctgatgggtaaaatcatcgactttgtggatatcctctccaaacccgaacgcattacccaaatcatccgtgacgaactggaagaaatcaaaaccaactatggcgacgaacgccgcagcgaaatcaacccgttcggcggcgacattgccgatgaagacctgattccgcaacgcgaaatggtcgtgaccctgacccacggcggctatataaaaacccagccgaccaccgactatcaggctcagcgtcgcggcgggcgcggcaaacaggcggctgccaccaaagacgaagactttatcgaaaccctgtttgttgccaacacgcatgactatttgatgtgttttaccaacctcggcaagtgccactggattaaggtttacaaactgcccgaaggcggacgcaacagccgcggccgtccgattaacaacgtcatccagctggaagaaggcgaaaaagtcagcgcgattctggcagtacgcgagtttcccgaagaccaatacgtcttcttcgccaccgcgcagggaatggtgaaaaaagtccaactttccgcctttaaaaacgtccgcgcccaaggcattaaagccatcgcactcaaagaaggcgactacctcgtcggcgctgcgcaaacaggcggtgcggacgacattatgttgttctccaacttgggcaaagccatccgcttcaacgaatactgggaaaaatccggcaacgacgaagcggaagatgccgacatcgaaaccgagatttcagacgacctcgaagacgaaaccgccgacaacgaaaacaccctgccaagcggcaaaaacggcgtgcgtccgtccggtcgcggcagcggcggtttgcgcggtatgcgcctgcctgccgacggcaaaatcgtcagcctgattaccttcgcccctgaaaccgaagaaagcggtttgcaagttttaaccgccaccgccaacggatacggaaaacgcaccccgattgccgattacagccgcaaaaacaaaggcgggcaaggcagtattgccattaacaccggcgagcgcaacggcgatttggtcgccgcaaccttggtcggcgaaaccgacgatttgatgctgattaccagcggcggcgtgcttatccgtaccaaagtcgaacaaatccgcgaaaccggccgcgccgcagcaggcgtgaaactgattaacttggacgaaggcgaaaccttggtatcgctggaacgtgttgccgaagacgaatccgaactctccggcgcttctgtaatttccaatgtaaccgaaccggaagccgagaactga

SEQ ID NO: 20 - protein, 916 aa, Neisseria gonorrhoeae
MTDATIRHDHKFALETLPVSLEDEMRKSYLDYAMSVIVGRALPDVRDGLKPVHRRVLYAMHELKNNWNAAYKKSARIVGDVIGKYHPHGDSAVYDTIVRMAQNFAMRYVLIDGQGNFGSVDGLAAAAMRYTEIRMAKISHEMLADIEEETVNFGPNYDGSEHEPLVLPTRFPTLLVNGSSGIAVGMATNIPPHNLTDTINACLRLLDEPKTEIDELIDIIQAPDFPTGATIYGLGGVREGYKTGRGRVVIRGKTHIEPIGKNGEREAIVIDEIPYQVNKAKLVEKIGDLVREKTLEGISELRDESDKSGMRVVIELKRNENAEVVLNQLYKLTPLQDSFGINMVVLVDGQPRLLNLKQILSEFLRHRREVVTRRTLFRLKKARHEGHIAEGKAVALSNIDEIIKLIKESPNAAEAKEKLLARPWRSSLVEEMLTRSGLDLEMMRPEGLAANIGLKKQGYYLSEIQADAILRMSLRNLTGLDQKEIIESYKNLMGKIIDFVDILSKPERITQIIRDELEEIKTNYGDERRSEINPFGGDIADEDLIPQREMVVTLTHGGYIKTQPTTDYQAQRRGGRGKQAAATKDEDFIETLFVANTHDYLMCFTNLGKCHWIKVYKLPEGGRNSRGRPINNVIQLEEGEKVSAILAVREFPEDQYVFFATAQGMVKKVQLSAFKNVRAQGIKAIALKEGDYLVGAAQTGGADDIMLFSNLGKAIRFNEYWEKSGNDEAEDADIETEISDDLEDETADNENTLPSGKNGVRPSGRGSGGLRGMRLPADGKIVSLITFAPETEESGLQVLTATANGYGKRTPIADYSRKNKGGQGSIAINTGERNGDLVAATLVGETDDLMLITSGGVLIRTKVEQIRETGRAAAGVKLINLDEGETLVSLERVAEDESELSGASVISNVTEPEAEN

SEQ ID NO: 21 - DNA, 2910 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgaagaagtgatgacgagtgtttacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgacttgtttattaaaaacacgagcactcttgccaacacgaaagtggacgtatagggtgtaacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtggaaggcttggaagcaaagacgccagtctctgtggagtcgtccttgaaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggcttgattccgcccaagagttcatatcgacggcggagtttggcacctcgatgtcggctcatcacatcctggggctgtagtcggtcccaagggtatggctgttcgccattttaaagtggtacgtgagttgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 22 - DNA, 2904 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataacccaagctgcgtatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgcaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 23 - DNA, 2903 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgaaccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 24 - DNA, 2903 bp, Neisseria gonorrhoeae
tgaaatgatagagtcaagtgaataagtgcatcaggcggatgccttggcgatgataggcgacgaaggacgtgtaagcctgcgaaaagcgcgggggagctggcaataaagcaatgatcccgcggtgtccgaatggggaaacccactgcattctgtgcagtatcctaagttgaatacataggcttagagaagcgaacccggagaactgacccatctaagtacccggaggaaaagaaatcaaccgagattccgcaagtagtggcgagcgaacgcggaggagcctgtacgtaataactgtcgaggtagaagaacaagctgggaagcttgaccatagcgggtgacagtcccgtattcgaaatctcaacagcggtactaagcgtacgaaaagtagggcgggacacgtgaaatcctgtctgaatatggggggaccatcctccaaggctaaatactcatcatcgaccgatagtgaaccagtaccgtgagggaaaggcgaaaagaaccccgggaggggagtgaaacagaacctgaaacctgatgcatacaaacagtgggagcgccctagtggtgtgactgcgtaccttttgtataatgggtcaacgacttacattcagtagcgagcttaaccggataggggaggcgtagggaaaccgagtcttaatagggcgatgagttgctgggtgtagacccgaaaccgagtgatctatccatggccaggttgaaggtgccgtaacaggtactggaggaccgaacccacgcatgttgcaaaatgcggggatgagctgtgggtaggggtgaaaggctaaacaaactcggagatagctggttctccccgaaaactatttaggtagtgcctcgagcaagacactgatgggggtaaagcactgttatggctagggggttattgcaacttaccaacccatggcaaactcagaataccatcaagtggttcctcgggagacagacagcgggtgctaacgtccgttgtcaagagggaaacaacccagaccgccggctaaggtcccaaatgatagattaagtggtaaacgaagtgggaaggcacagacagccaggatgttggcttagaagcagccatcatttaaagaaagcgtaatagctcactggtcgagtcgtcctgcgcggaagatgtaacggggctcaaatctataaccgaagctgcggatgccggtttaccggcatggtaggggagcgttctgtaggctgatgaaggtgcattgtaaagtgtgctggaggtatcagaagtgcgaatgttgacatgagtagcgataaagcgggtgaaaagcccgctcgccgaaagcccaaggtttcctacgcaacgttcatcggcgtagggtgagtcggcccctaaggcgaggcagaaatgcgtagtcgatgggaaacaggttaatattcctgtacttgattcaaatgcgatgtggggacggagaaggttaggttggcaagctgttggaatagcttgtttaagccggtaggtggaagacttaggcaaatccgggttttcttaacaccgagaagtgatgacgagtgtctacggacacgaagcaaccgataccacgcttccaggaaaagccactaagcttcagtttgaatcgaaccgtaccgcaaaccgacacaggtgggcaggatgagaattctaaggcgcttgagagaactcgggagaaggaactcggcaaattgataccgtaacttcgggagaaggtatgccctctaaggttaaggacttgctccgtaagccccggagggtcgcagagaataggtggctgcgactgtttattaaaaacacagcactctgccaacacgaaagtggacgtatagggtgtgacgcctgcccggtgccggaaggttaattgaagatgtgcaagcatcggatcgaagccccggtaaacggcggccgtaactataacggtcctaaggtagcgaaattccttgtcgggtaagttccgacccgcacgaatggcgtaacgatggccacactgtctcctcccgagactcagcgaagttgaagtggttgtgaagatgcaatctacccgctgctagacggaaagaccccgtgaacctttactgtagctttgcattggactttgaagtcacttgtgtaggataggtgggaggcttggaagcagagacgccagtctctgtggagtcgtccttgaaataccaccctggtgtctttgaggttctaacccagacccgtcatccgggtcggggaccgtgcatggtaggcagtttgactggggcggtctcctcccaaagcgtaacggaggagttcgaaggttacctaggtccggtcggaaatcggactgatagtgcaatggcaaaaggtagcttaactgcgagaccgacaagtcgggcaggtgcgaaagcaggacatagtgatccggtggttctgtatggaagggccatcgctcaacggataaaaggtactccggggataacaggctgattccgcccaagagttcatatcgacggcggagtttggcacctcgatgtcggctcatcacatcctggggctgtagtcggtcccaagggtatggctgttcgccatttaaagtggtacgtgagctgggtttaaaacgtcgtgagacagtttggtccctatctgcagtgggcgttggaagtttgacgggggctgctcctagtacgagaggaccggagtggacgaacctctggtgtaccggttgtaacgccagttgcatagccgggtagctaagttcggaagagataagcgctgaaagcatctaagcgcgaaactcgcctgaagatgagacttcccttgcggtttaaccgcactaaagggtcgttcgagaccaggacgttgataggtggggtgtggaagcgcggtaacgcgtgaagctaacccatactaattgcccgtgaggcttgactct

SEQ ID NO: 25 - DNA, 633 bp, Neisseria gonorrhoeae
atgagaaaaaccaaaaccgaagccttgaaaaccaaagaacacctgatgcttgccgccttggaaaccttttaccgcaaagggattgcccgcacctcgctcaacgaaatcgcccaagccgccggcgtaacgcgcggcgcgctctattggcatttcaaaaataaggaagacttgtttgacgcgttgttccaacgtatctgcgacgacatcgaaaactgcatcgcgcaagatgccgcagatgccgaaggaggttcttggacggtattccgccacacgctgctgcactttttcgagcggctgcaaagcaacgacatccactacaaattccacaacatcctgtttttaaagtgcgaacatacggaacaaaacgccgccgttatcgccattgcccgcaagcatcaggcaatctggcgcgagaaaattaccgccgttttgaccgaagcggtggaaaatcaggatttggctgacgatttggacaaggaaacggcggtcatcttcatcaaatcgacgttggacgggctgatttggcgttggttctcttccggcgaaagtttcgatttgggcaaaaccgccccgcgcatcatcgggataatgatggacaacttggaaaaccatccctgcctgcgccggaaataa

SEQ ID NO: 26 - protein, 210 aa, Neisseria gonorrhoeae
MRKTKTEALKTKEHLMLAALETFYRKGIARTSLNEIAQAAGVTRGALYWHFKNKEDLFDALFQRICDDIENCIAQDAADAEGGSWTVFRHTLLHFFERLQSNDIHYKFHNILFLKCEHTEQNAAVIAIARKHQAIWREKITAVLTEAVENQDLADDLDKETAVIFIKSTLDGLIWRWFSSGESFDLGKTAPRIIGIMMDNLENHPCLRRK

SEQ ID NO: 27 - DNA, 29 bp, Neisseria gonorrhoeae
ttgcacggataaaaagtcttttttataat